(19) United States
(12) Patent Application Publication
(10) Pub. No.: US 2004/0146509 A1
(43) Pub. Date: Jul. 29, 2004

(54) Methods for improvement of lung function using TGF-beta inhibitors

(76) Inventors: Zhihe Li, Foster City, CA (US); David Y. Liu, Palo Alto, CA (US); Jing Ying Ma, Cupertino, CA (US); Andrew A. Protter, Palo Alto, CA (US); George F. Schreiner, Los Altos, CA (US); Thomas-Toan Tran, Sunnyvale, CA (US)

Correspondence Address: GINGER R. DREGER, ESQ., Heller, Ehrman White & McAuliffe, LLP, 275 Middlefield Road, Menlo Park, CA, 94025, US

(21) Appl. No.: 10/626,004

(22) Filed: Jul. 23, 2003

Related U.S. Application Data
(60) Provisional application No. 60/399,369, filed on Jul. 25, 2002.
(60) Provisional application No. 60/399,368, filed on Jul. 25, 2002.

(51) Int. Cl. A61K 39/395

(52) U.S. Cl. 424/144.1

(57) ABSTRACT

The invention concerns methods for improvement of lung function by administering non-peptide small molecule inhibitors of TGF-β specifically binding to the type I TGF-β receptor (TGFβ-R1). Preferably, the inhibitors are quinazoline derivatives.

### CROSS-REFERENCE TO RELATED APPLICATIONS

This is a non-provisional application filed under 37 CFR 1.53(b), claiming priority under USC Section 119(e) to provisional Application Ser. No. 60/399,368 filed Jul. 25, 2002.

### BACKGROUND OF THE INVENTION

1. Field of the Invention

The present invention concerns methods of treatment using transforming growth factor β (TGF-β) inhibitors. More specifically, the invention concerns methods of improving lung function by administering TGF-β inhibitors that inhibit biological activities mediated by the type I TGF-β receptor (TGFβ-R1).

2. Description of the Related Art

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). Other members of this superfamily include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication.

TGF-β exerts its biological activities through a receptor system including the type I and type II single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-R1) by the type II receptor (TGFβ-R2). The activated TGFβ-R1 phosphorylates a receptor-associated co-transcription factor Smad2/Smad3, thereby releasing it into the cytoplasm, where it binds to Smad4. The Smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. 75:111-57 (2000), which are expressly incorporated herein by reference.

### SUMMARY OF THE INVENTION

The invention concerns a method for the improvement of lung function, comprising the administration, to a mammalian subject diagnosed with a disease or condition benefiting from the improvement of lung function, an effective amount of a molecule capable of inhibiting a biological activity mediated by a TGFβ-R1 kinase receptor.

The invention further concerns a method for the treatment of a mammalian subject having impaired lung function, comprising administering to such subject an effective amount of a molecule capable of inhibiting a biological activity mediated by a TGFβ-R1 kinase receptor.

The subject preferably is human.

In a particular embodiment, the molecule is a TGF-β inhibitor specifically binding to a TGFβ-R1 kinase receptor. In another particular embodiment, the molecule is a non-peptide small molecule, e.g. a small organic molecule.

The disease or condition benefiting from the improvement of lung function may, for example, be selected from the group consisting of emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, lung injuries resulting from infectious causes, inhaled toxins, or circulating exogenous toxins, aging and genetic predisposition to impaired lung function.

In a further embodiment, the small molecule inhibitor additionally inhibits a biological activity mediated by p38 kinase.

In another embodiment, the small molecule inhibitor preferentially inhibits a biological activity mediated by TGF-β-R1 kinase relative to a biological activity mediated by p38 kinase.

In a further embodiment, the small molecule inhibitor is other than an imidazole derivative.

In a still further embodiment, the small molecule inhibitor is a compound of formula (1)

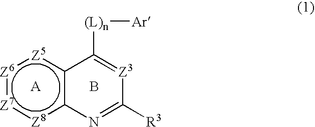

or the pharmaceutically acceptable salts thereof

wherein R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

In a preferred embodiment, the compound of formula (1) is a quinazoline derivative.

In another preferred group of compounds of formula (1) Z³ is N; and Z⁵-Z⁸ are CR².

In a different group, Z³ is N; and at least one of Z⁵-Z⁸ is nitrogen. Compounds in which R³ is an optionally substituted phenyl moiety are specifically included.

Another group of compounds for use in the methods of the present invention is represented by the following formula (2)

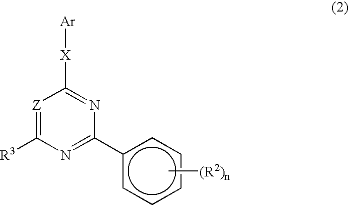

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

Another group of the compounds of the invention is represented by formula (3)

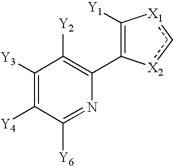

wherein Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₙ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S:

Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one o more substituents independently selected from alkyl(1-6 C), alkoxy(a-6 C), haloalkyl(1-6 C), halo, NH₂, NH-alkyl(1-6 C), or NH(CH₂)ₙ—Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ—Ph wherein n is 0-3; and

one of X₁ and X₂ is N and the other is NR₆, wherein R₆ is hydrogen or alkyl(1-6 C).

As used in formula (3), the double bonds indicated by the dotted lined represent possible tautomeric ring forms of the compounds. Further information about compounds of formula (3) and their preparation is disclosed in WO 02/40468, published May 23, 2002, the entire disclosure of which is hereby expressly incorporated by reference.

Yet another group of compounds for use in the methods of the invention is represented by the following formula (4)

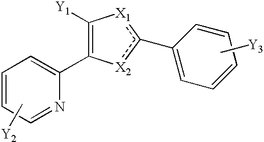

wherein Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)—Ph, —S—(CH₂)ₙ—Ph, cyano, phenyl, and CO₂R, wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;

Y₂ is H, NH(CH₂)ₙ—Ph or NH-alkyl(1-6 C), wherein n is 0, 1, 2, or 3;

Y₃ is CO₂H, CONH₂, CN, NO₂, alkylthio(1-6 C), —SO₂-alkyl(C1-6), alkoxy(C1-6), SONH₂, CONHOH, NH₂, CHO, CH₂NH₂, or CO₂R, wherein R is hydrogen or alkyl(1-6 C);

one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Pharmaceutically acceptable salts of all compounds within the scope of the invention are specifically included.

### BRIEF DESCRIPTION OF THE DRAWINGS

FIG. 1 shows the effect of a representative compound of formula (1) on the respiratory rate in a 5-day bleomycin rat lung injury model.

FIG. 2 shows the effect of a representative compound of formula (1) on the tidal volume in a 5-day bleomycin rat lung injury model.

FIG. 3 shows the effect of a representative compound of formula (1) on the total BALF IL-6 in a 5-day bleomycin rat lung injury model.

FIG. 4 shows the effect of a representative compound of formula (1) on total lung capacity in a 5-day bleomycin rat lung injury model.

FIG. 5 shows the effect of a representative compound of formula (1) on permeability in a 5-day bleomycin rat lung injury model.

FIG. 6 illustrates that treatment with a representative compound of formula (1) reduces lung permeability as measured by fluorescence following RITC-Dextran administration to rats with bleomycin-induced lung injury.

FIG. 7 shows that treatment with a representative compound of formula (1) reduces tissue damage in bleomycin 5 day rat lung injury model.

FIG. 8 shows the effect of a representative compound of formula (1) on lung hydroxyproline content following bleomycin-induced lung fibrosis.

FIG. 9 shows the effect of a representative compound of formula (1) on total lung capacity following bleomycin-induced lung fibrosis.

FIG. 10 shows that a representative compound of formula (1) significantly reduces lung fibrosis induced by bleomycin.

FIGS. 11 and 12 are histology pictures showing that treatment with a representative compound of formula (1) reduces fibrosis in the 14-day bleomycin rat lung injury model.

### DETAILED DESCRIPTION OF THE PREFERRED EMBODIMENT

A. Definitions

The terms “improvement of lung function,” and “improvement of pulmonary function” are used interchangeably, and refer to an improvement in any parameter suitable to measure lung performance. Thus, improvement of pulmonary function can be measured, for example, in murine bleomycin-induced lung injury models, such as the bleomycin rat lung injury model described in the Examples below, which monitors improvements in respiratory rate and tidal volume. Parameters that are typically monitored in human patients as a measure of lung function include, but are not limited to, inspiratory and expiratory flow rates, lung volume (also referred to as lung capacity), and diffusing capacity for carbon monoxide, ability to forcibly exhale, respiratory rate, and the like. Methods of quantitatively determining pulmonary function in patients are well known in the art, and include timed measurement of inspiratory and expiratory maneuvers to measure specific parameters. For example, forced vital capacity (FVC) measures the total volume in liters exhaled by a patient forcefully from a deep initial inspiration. This parameter, when evaluated in conjunction with the forced expired volume in one second (FEV₁), allows bronchoconstriction to be quantitatively evaluated. In addition to measuring volumes of exhaled air as indices of pulmonary function, the flow in liters per minute measured over differing portions of the expiratory cycle can be useful in determining the status of a patient's pulmonary function. In particular, the peak expiratory flow, taken as the highest air flow rate in liters per minute during a forced maximal exhalation, is well correlated with overall pulmonary function in a patient with respiratory diseases. Methods and tools for measuring these and similar parameters are well known in the art, and routinely used in everyday clinical practice.

The term “tidal volume” refers to the volume of air inspired or expired with each normal breath.

A “biological activity mediated by the TGFβ-R1 kinase receptor” can be any activity associated with the activation of TGFβ-R1 and downsteam intracellular signaling events, such as the phosphorylation of Smad2/Smad3.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. Thus, in the context of improving lung function, treatment includes prevention and treatment of a disease or condition negatively impacting lung function or otherwise benefiting from the improvement of lung function, relieving one or more symptoms of such disease, prevention and treatment of complications resulting from such disease, improving exercise tolerance of patients with compromised lung function, and reduction in mortality.

The “pathology” of a disease or condition negatively impacting lung function includes all phenomena that compromise the well-being of the patient.

A “disease or condition benefiting from the improvement of lung function” includes all diseases, disorders and conditions which involve a negative change in at least one parameter suitable for measurement of lung performance. Such diseases and conditions include, without limitation, emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, and lung injuries resulting from other factors, such as, infectious causes, inhaled toxins, or circulating exogenous toxins, aging and genetic predisposition to impaired lung function.

The term “inhibitor” as used herein refers to a molecule, e.g. a nonpeptide small molecule, having the ability to inhibit the biological function of a native TGF-β molecule mediated by the TGFβ-R1 receptor. Accordingly, the term “inhibitor” is defined in the context of the biological role of TGF-β and its receptors. Preferred inhibitors within the scope of the invention specifically bind a TGFβ-R1 receptor. Other preferred inhibitors preferentially inhibit the function of a TGFβ-R1 receptor through specific binding to that receptor or otherwise.

The terms “specifically binding,” “binds specifically,” “specific binding,” and grammatical equivalents thereof, are used to refer to binding to a unique epitope within the type I TGF-β receptor (TGFβ-R1). The binding must occur with an affinity to effectively inhibit TGF-β signaling through TGFβ-R1.

The term “preferentially inhibit” as used herein means that the inhibitory effect on the target that is “preferentially inhibited” is significantly greater than on any other target. Thus, in the context of preferential inhibition of TGF-β-R1 kinase relative to the p38 kinase, the term means that the inhibitor inhibits biological activities, e.g. profibrotic activities, mediated by the TGF-β-RL kinase significantly more than biological activities mediated by the p38 kinase. The difference in the degree of inhibition, in favor of the preferentially inhibited receptor, generally is at least about two-fold, more preferably at least about five-fold, even more preferably at least about ten-fold.

The term “mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

A “therapeutically effective amount”, in reference to the treatment of a disease, e.g. when inhibitors of the present invention are used, refers to an amount capable of invoking one or more of the following effects: (1) inhibition (i.e., reduction, slowing down or complete stopping) of the development or progression of a disease or condition negatively affecting lung function; (2) inhibition (i.e., reduction, slowing down or complete stopping) of consequences of or complications resulting from such disease or condition; and (3) relief, to some extent, of one or more symptoms associated with such disease or condition, or symptoms of consequences of or complications resulting from such disease and/or condition.

As used herein, a “noninterfering substituent” is a substituent which leaves the ability of the compound of formula (1) to inhibit TGF-β activity qualitatively intact. Thus, the substituent may alter the degree of inhibition. However, as long as the compound of formula (1) retains the ability to inhibit TGF-β activity, the substituent will be classified as “noninterfering.”

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety refers to a monocyclic or fused bicyclic moiety such as phenyl or naphthyl; “heteroaromatic” also refers to monocyclic or fused bicyclic ring systems containing one ore more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C. These carbon chains may also include a carbonyl group, thus making them able to provide substituents as an acyl moiety.

B. Modes of Carrying out the Invention

As discussed before, the biological activities of TGF-β are mediated by two distinct types of receptors designated type I and type II (Derynck and Feng, Biochim. Biophys. Acta 1333:F105-F150 (1997); Massague, Annu. Rev. Biochem., 67:753-91 (1998)). Both receptors are serine-threonine kinases. Upon binding of TGF-β to the type II receptor, the type II receptor phosphorylates the type I receptor, which is activated and is, in turn, responsible for intracellular signaling. In addition, TGF-β has a non-serine-theronine kinase receptor, termed type I/II receptor, which is believed to facilitate or modulate signaling through the type I/II receptor pair (Lopez-Casillas et al., Cell 73:996-1005 (1993)).

The present invention is based on the surprising finding that certain quinazoline and imidazole derivatives specifically inhibiting TGF-β signaling through the type I TGF-β receptor (TGFβ-R1), e.g. by specifically binding TGFβ-R1, can improve lung function.

In a preferred embodiment, the inhibitors of the present invention selectively inhibit biological responses mediated by the type I receptor, without affecting the type II receptor-mediated cell proliferation.

In another preferred embodiment, the compounds of the present invention preferentially inhibit TGFβ-R1 kinase relative to p38 kinase.

Compounds of the Invention

The inhibitors of the present invention typically are small organic molecules (non-peptide small molecules), generally less than about 1,000 daltons in size. Preferred non-peptide small molecules have molecular weights of less than about 750, daltons, more preferably less than about 500 daltons, and even more preferably less than about 300 daltons.

In a preferred embodiment, the compounds of the invention are of the formula

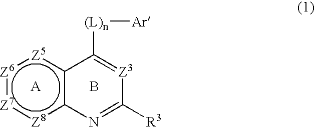

or the pharmaceutically acceptable salts thereof

wherein R³ is a noninterfering substituent;

each Z is CR² or N, wherein no more than two Z positions in ring A are N, and wherein two adjacent Z positions in ring A cannot be N;

each R² is independently a noninterfering substituent;

L is a linker;

n is 0 or 1; and

Ar′ is the residue of a cyclic aliphatic, cyclic heteroaliphatic, aromatic or heteroaromatic moiety optionally substituted with 1-3 noninterfering substituents.

In a preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z⁶ and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8 Å, preferably 2-6 Å, more preferably 2-4 Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)₁, NR¹(CR²₂)ₘ, NR¹CO(CR²₂)₁, O(CR²₂)ₘ, OCO(CR²₂)₁, and

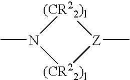

wherein Z is N or CH and wherein m is 0-4 and 1 is 0-3, preferably 1-3 and 1-2, respectively. L preferably provides —NR¹— coupled directly to ring B. A preferred embodiment of R¹ is H, but R¹ may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted or unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³ is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR2, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro-4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent. As set forth above, a “noninterfering substituent” is one whose presence does not substantially destroy the TGF-β inhibiting ability of the compound of formula (1).

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR2, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alkyl, alkenyl, alkynyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR⁴₂, SR⁴, —OOCR⁴, —NROCR⁴, —COOR⁴, R⁴CO, —CONR⁴₂, —SO₂NR⁴₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R₂ may also, itself, be selected from the group consisting of halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C).

More preferred substituents represented by R² are those as set forth with regard to the phenyl moieties contained in Ar′ or R³ as set forth above. Two adjacent CR² taken together may form a carbocyclic or heterocyclic fused aliphatic ring of 5-7 atoms. Preferred R² substituents are of the formula R⁴, —OR⁴, SR⁴ or R⁴NH—, especially R⁴NH—, wherein R⁴ is defined as above. Particularly preferred are instances wherein R⁴ is substituted arylalkyl. Specific representatives of the compounds of formula (1) are shown in Tables 1-3 below. All compounds listed in Table 1 have a quinazoline ring system (Z³ is N), where the A ring is unsubstituted (Z⁵-Z⁸ represent CH). The substituents of the B ring are listed in the following Table 1.

TABLE 1

| Compound N. | L | Ar′ | R³ |
|---|---|---|---|
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | phenyl |
| 12^{†} | N^{†} | 4-pyridyl | phenyl |
| 13 | NHCH₂ | 4-pyridyl | phenyl |
| 14 | NHCH₂ | 4-pyridyl | 4-chlorophenyl |
| 15 | NH | 3-pyridyl | phenyl |
| 16 | NHCH₂ | 2-pyridyl | phenyl |
| 17 | NHCH₂ | 3-pyridyl | phenyl |
| 18 | NHCH₂ | 2-pyridyl | phenyl |
| 19 | NHCH₂CH₂ | 2-pyridyl | phenyl |
| 20 | NH | 6-pyrimidinyl | phenyl |
| 21 | NH | 2-pyrimidinyl | phenyl |
| 22 | NH | phenyl | phenyl |
| 23 | NHCH₂ | phenyl | 3-chlorophenyl |
| 24 | NH | 3-hydroxyphenyl | phenyl |
| 25 | NH | 3-hydroxyphenyl | phenyl |
| 26 | NH | 4-hydroxyphenyl | phenyl |
| 27 | NH | 4-indolyl | phenyl |
| 28 | NH | 5-indolyl | phenyl |
| 29 | NH | 4-methoxyphenyl | phenyl |
| 30 | NH | 3-methoxyphenyl | phenyl |
| 31 | NH | 2-methoxyphenyl | phenyl |
| 32 | NH | 4-(2-hydroxyethyl)phenyl | phenyl |
| 33 | NH | 3-cyanophenyl | phenyl |
| 34 | NHCH₂ | 2,5-difluorophenyl | phenyl |
| 35 | NH | 4-(2-butyl)phenyl | phenyl |
| 36 | NHCH₂ | 4-dimethylaminophenyl | phenyl |
| 37 | NH | 4-pyridyl | cyclopentyl |
| 38 | NH | 2-pyridyl | phenyl |
| 39 | NHCH₂ | 3-pyridyl | phenyl |
| 40 | NH | 4-pyrimidyl | phenyl |
| 41^{‡} | NH^{‡} | 4-pyridyl | phenyl |
| 42 | NH | p-aminomethylphenyl | phenyl |
| 43 | NHCH₂ | 4-aminophenyl | phenyl |
| 44 | NH | 4-pyridyl | 3-chlorophenyl |
| 45 | NH | phenyl | 4-pyridyl |
| 46 | NH | 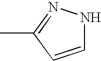 | phenyl |
| 47 | NH | 4-pyridyl | t-butyl |
| 48 | NH | 2-benzylamino-3-pyridyl | phenyl |
| 49 | NH | 2-benzylamino-4-pyridyl | phenyl |
| 50 | NH | 3-benzyloxyphenyl | phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 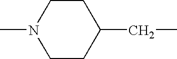 | 4-pyridyl | phenyl |
| 55 | 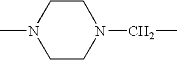 | phenyl | phenyl |
| 56 | 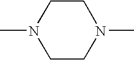 | 2-pyridyl | phenyl |
| 57 | NHCH₂CH₂ | 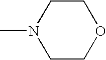 | phenyl |
| 58 | not present | 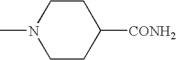 | phenyl |
| 59 | not present | 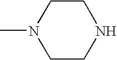 | phenyl |
| 60 | NH | 4-pyridyl | cyclopropyl |
| 61 | NH | 4-pyridyl | 2-trifluoromethylphenyl |
| 62 | NH | 4-aminophenyl | phenyl |
| 63 | NH | 4-pyridyl | cyclohexyl |
| 64 | NH | 3-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 3-amino-4-pyridyl | phenyl |
| 68 | NH | 4-pyridyl | 2-benzylaminophenyl |
| 69 | NH | 2-benzylaminophenyl | phenyl |
| 70 | NH | 2-benzylaminophenyl | 4-cyanophenyl |
| 71 | NH | 3′-cyano-2-benzylaminophenyl | phenyl |

*R¹ = 2-propyl
^{†}R¹ = 4-methoxyphenyl
^{‡}R¹ = 4-methoxybenzyl

The compounds in Table 2 contain modifications of the quinazoline nucleus as shown. All of the compounds in Table 2 are embodiments of formula (I) wherein Z³ is N and Z⁶ and Z⁷ represent CH. In all cases the linker, L, is present and is NH.

TABLE 2

| Compound N | Z⁵ | Z⁶ | Ar′ | R³ |
|---|---|---|---|---|
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)-pyridyl | 5-chloro-2- |
| | | | | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)-pyridyl | 5-chloro-2- |
| | | | | fluorphenyl |

Additional compounds were prepared wherein ring A contains CR² at Z⁶ or Z⁷ where R² is not H. These compounds, which are all quinazoline derivatives, wherein L is NH and Ar′ is 4-pyridyl, are shown in Table 3.

TABLE 3

| Compound | | |
|---|---|---|
| No. | R³ | CR² as noted |
| 81 | 2-chlorophenyl | 6,7-dimethoxy |
| 82 | 2-fluorophenyl | 6-nitro |
| 83 | 2-fluorophenyl | 6-amino |
| 84 | 2-fluorophenyl | 7-amino |
| 85 | 2-fluorophenyl | 6-(3-methoxybenzylamino) |
| 86 | 2-fluorophenyl | 6-(4-methoxybenzylamino) |
| 87 | 2-fluorophenyl | 6-(2-isobutylamino) |
| 88 | 2-fluorophenyl | 6-(4- |
| | | methylmercaptobenzylamino) |
| 89 | 2-fluorophenyl | 6-(4-methoxybenzoyl amino) |
| 90 | 4-fluorophenyl | 7-amino |
| 91 | 4-fluorophenyl | 7-(3-methoxybenzylamino) |

Structures representative of quinazoline derivatives are shown below in Table 4.

TABLE 4

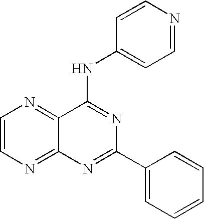

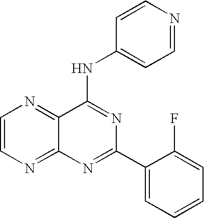

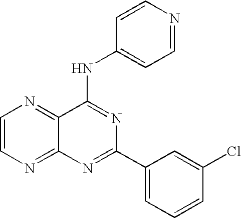

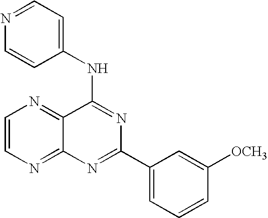

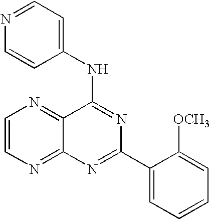

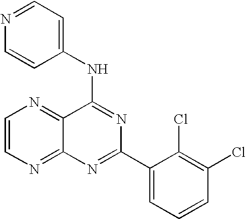

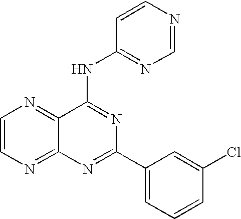

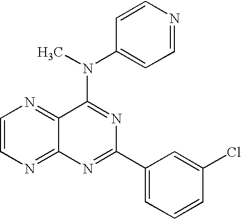

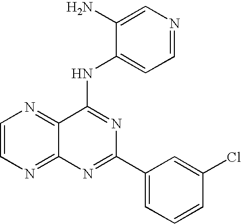

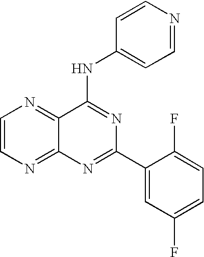

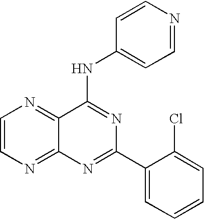

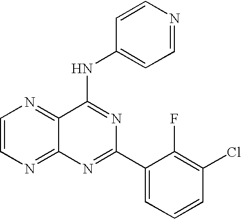

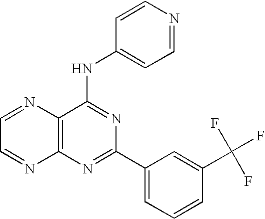

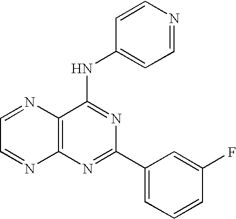

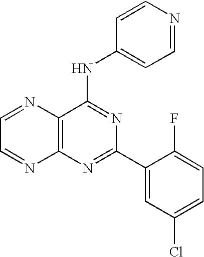

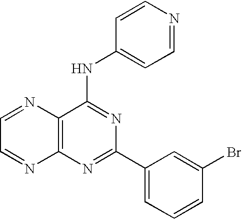

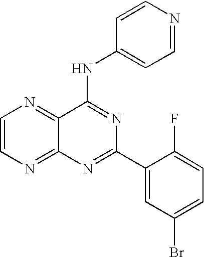

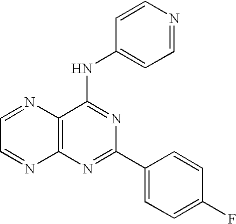

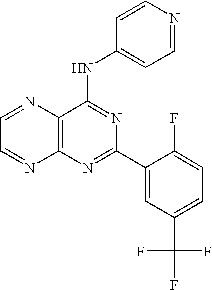

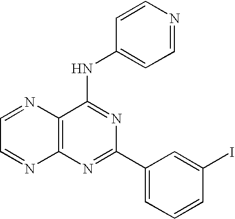

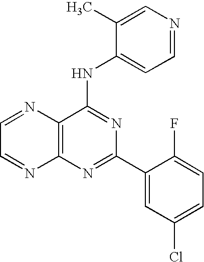

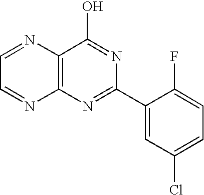

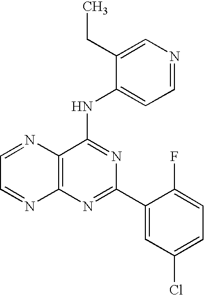

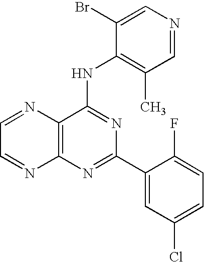

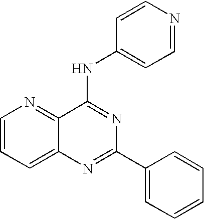

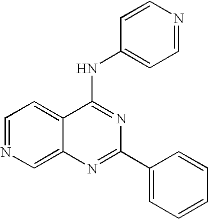

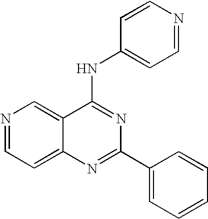

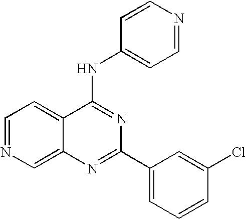

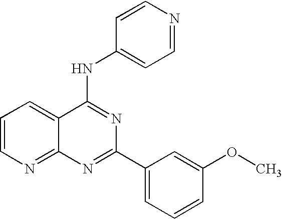

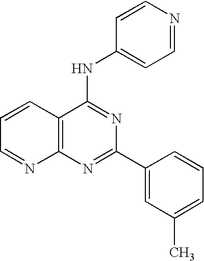

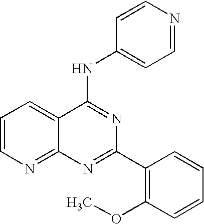

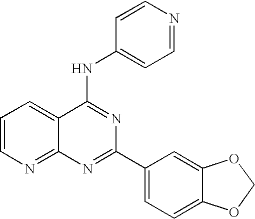

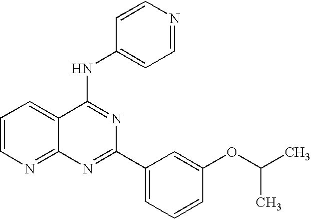

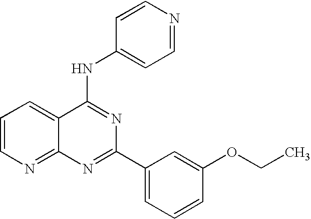

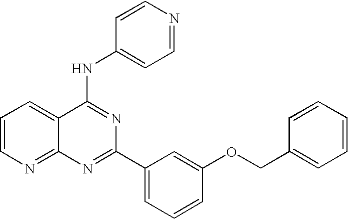

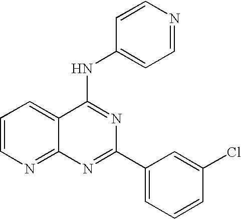

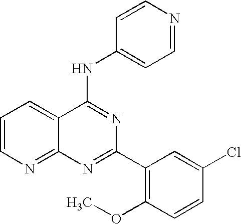

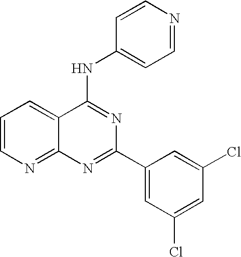

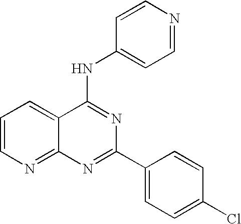

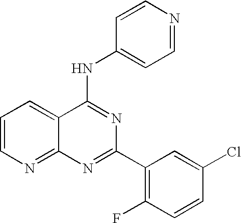

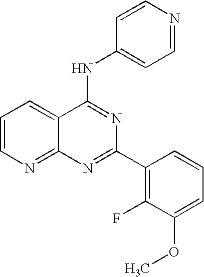

Although the invention is illustrated with reference to certain quinazoline derivatives, it is not so limited. Inhibitors of the present invention include compounds having a non-quinazoline, such as, a pyridine, pyrimidine nucleus carrying substituents like those discussed above with respect to the quinazoline derivatives.

Another group of compounds for use in the methods of the present invention is represented by the following formula (2)

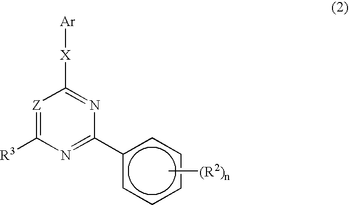

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R¹ is H, alkyl (1-8C), alkenyl (2-8C), or alkynyl (2-8C); or

Z represents N or CR⁴; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or

n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

Because tautomers are theoretically possible, phthalimido is also considered aromatic, and phthalimido-substituted alkyl and phthalimido-substituted alkoxy are preferred embodiments of R³ and R⁴.

In general, any alkyl, alkenyl, alkynyl, acyl, or aryl group contained in a substituent may itself optionally be substituted by additional substituents. The nature of these substituents is similar to those recited with regard to the primary substituents themselves. Thus, where an embodiment of, for example, R⁴ is alkyl, this alkyl may optionally be substituted by the remaining substituents listed as embodiments for R⁴ where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included within the scope of the invention. The features of the compounds are defined by formula (2) and the nature of the substituents is less important as long as the substituents do not interfere with the stated biological activity of this basic structure.

Non-interfering substituents embodied by R², R³ and R⁴, include, but are not limited to, alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, wherein each R is independently H or alkyl (1-8C), —CN, —CF₃, and NO₂, and like substituents. R³ and R⁴ can also be H. Preferred embodiments for R³ and R⁴ are H, alkyl (1-10C) or a heteroatom-containing form thereof, each optionally substituted, especially (1-4C) alkyl; alkoxy (1-8C), acylamido, aryloxy, arylalkyloxy, especially wherein the aryl group is a phthalimido group, and alkyl or arylalkyl amine. Preferred embodiments of R² include lower alkyl, alkoxy, and halo, preferably halo. Halo, as defined herein includes fluoro, chloro, bromo and iodo. Fluoro and chloro are preferred.

Preferably, R¹ is H or lower alkyl (1-4C), more preferably H.

Preferably Ar is optionally substituted phenyl, 2-, 3- or 4-pyridyl, indolyl, 2- or 4-pyrimidyl, pyridazinyl, benzotriazol or benzimidazolyl. More preferably Ar is phenyl, pyridyl, or pyrimidyl. Each of these embodiments may optionally be substituted with a group such as alkyl, alkenyl, alkynyl, aryl, O-aryl, O-alkylaryl, O-aroyl, NR-aryl, N-alkylaryl, NR-aroyl, halo, OR, NR₂, SR, —OOCR, —NROCR, RCO, —COOR, —CONR₂, and/or SO₂NR₂, wherein each R is independently H or alkyl (1-8C), and/or by —CN, —CF₃, and/or NO₂. Alkyl, alkenyl, alkynyl and aryl portions of these may be further substituted by similar substituents.

Preferred substituents on Ar include alkyl, alkenyl, alkynyl, halo, OR, SR, NR₂ wherein R is H or alkyl (1-4C); and/or arylamino, arylalkylamino, including alkylamino which is substituted by more than one aryl. As stated above, any aryl or alkyl group included within a substituent may itself be substituted similarly. These substituents may occupy all available positions of the ring, preferably 1-2 positions, or more preferably only one position.

Any of the aryl moieties, including those depicted in formula (2) especially the phenyl moieties, may also comprise two substituents which, when taken together, form a 5-7 membered carbocyclic or heterocyclic aliphatic ring. Similarly, R⁴ may be bridged to R³ to obtain a 5-7 membered carbocyclic or heterocyclic ring.

Structures representative of pyrimidine derivatives are shown below in Table 5.

TABLE 5

| | |
|---|---|
| I. | 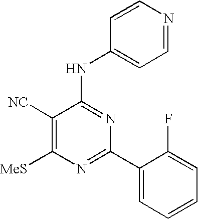 |
| II. | 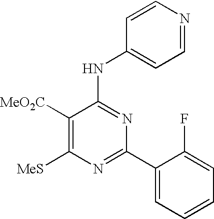 |
| II. | 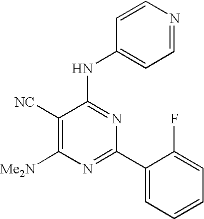 |
| IV. | 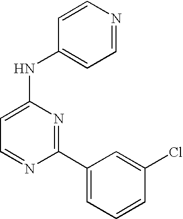 |
| V. | 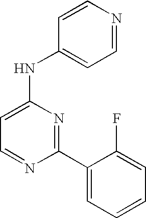 |
| VI. | 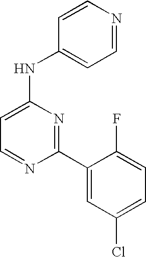 |
| VII. | 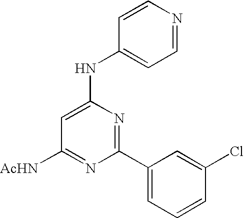 |
| VIII. | 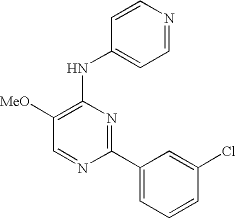 |
| IX | 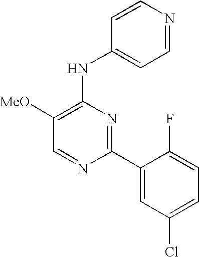 |
| X. | 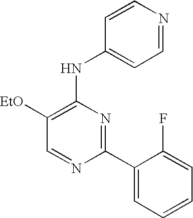 |
| XI. | 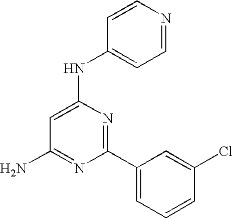 |
| XII. | 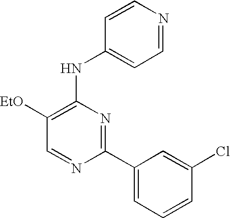 |
| XIII. | 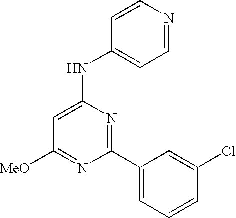 |
| XIV. | 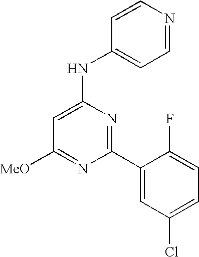 |
| XV. | 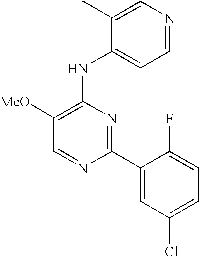 |
| XVI. | 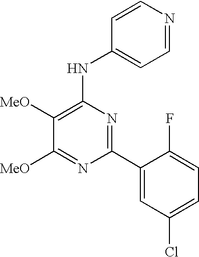 |
| XVII. | 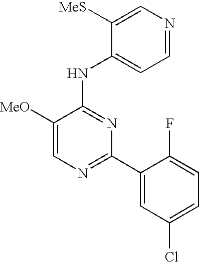 |
| XVIII. | 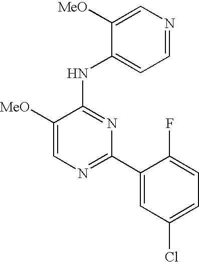 |
| XIX. | 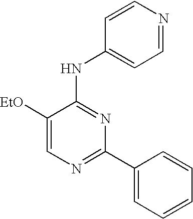 |
| XX. | 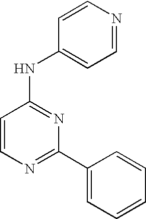 |
| XXI. | 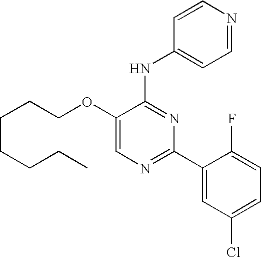 |
| XXII. | 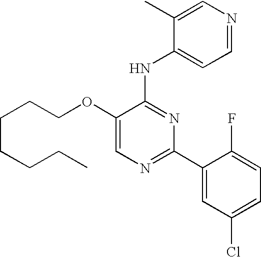 |
| XXIII. | 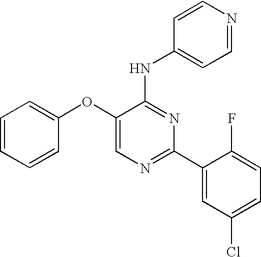 |
| XXIV. | 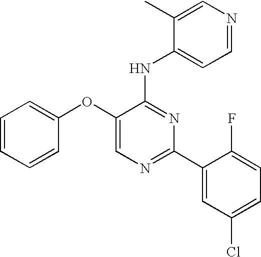 |
| XXV. | 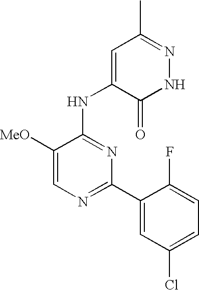 |
| XXVI. | 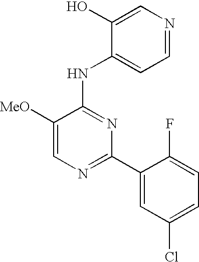 |
| XXVII. | 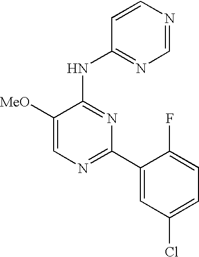 |
| XXVIII. | 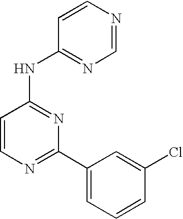 |
| XXIX. | 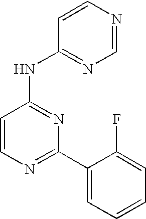 |
| XXX. | 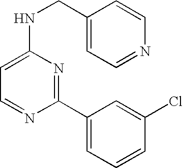 |
| XXXI. | 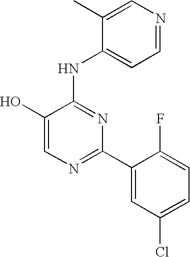 |
| XXXII. | 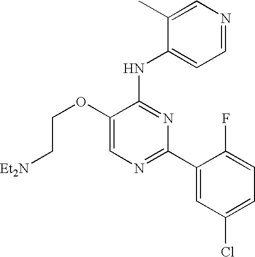 |
| XXXIII. | 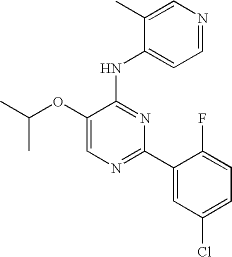 |
| XXXIV. | 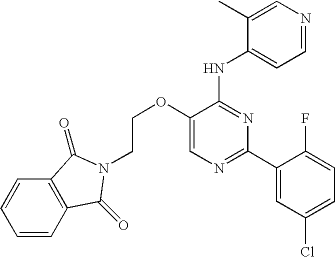 |
| XXXV. | 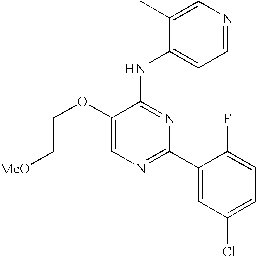 |
| XXXVI. | 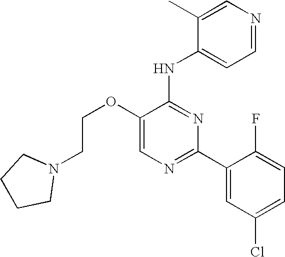 |
| XXXVII. | 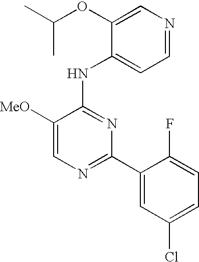 |
| XXXVIII. | 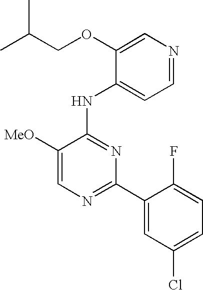 |
| XXXIX. | 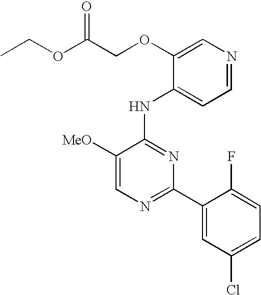 |
| XL. | 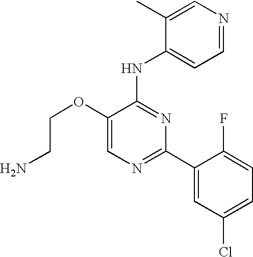 |
| XLI. | 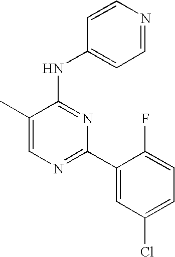 |
| XLII. | 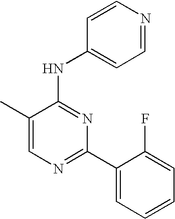 |
| XLIII. | 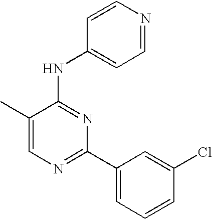 |
| XLIV. | 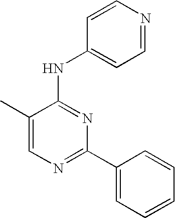 |
| XLV. | 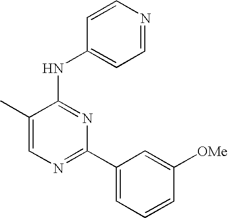 |
| XLVI. | 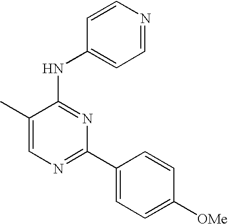 |
| XLVII. | 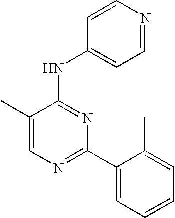 |
| XLVIII. | 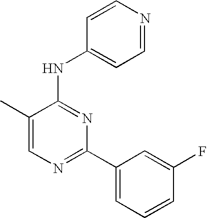 |
| XLIX. | 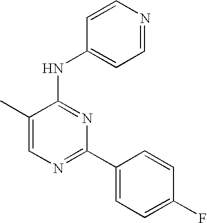 |
| L. | 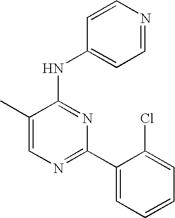 |
| LI. | 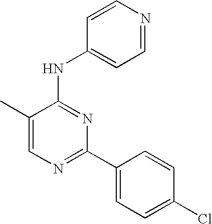 |
| LII. | 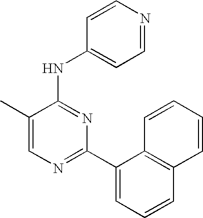 |
| LIII. | 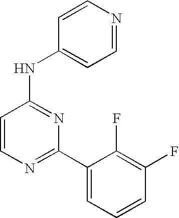 |
| LIV. | 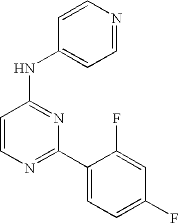 |
| LV. | 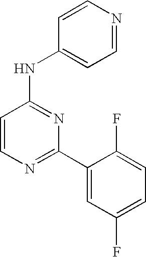 |
| LVI. | 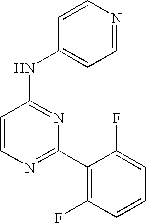 |
| LVII. | 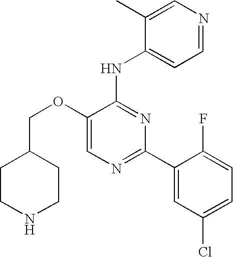 |
| LVIII. | 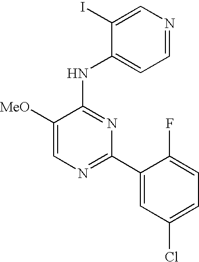 |
| LIX. | 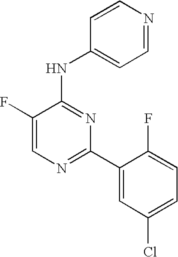 |
| LX. | 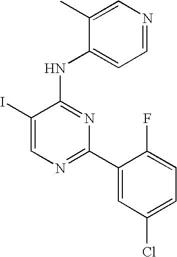 |
| LXI. | 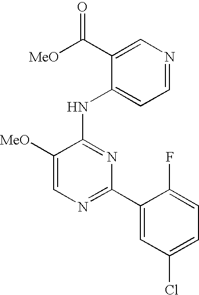 |
| LXII. | 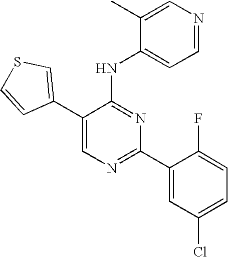 |
| LXIII. | 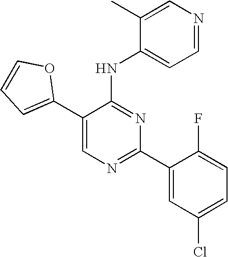 |
| LXIV. | 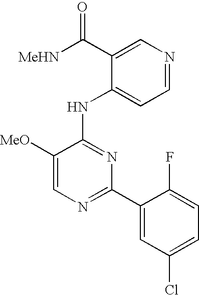 |
| LXV. | 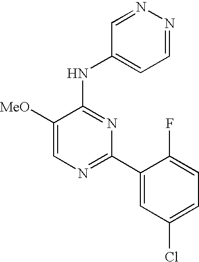 |
| LXVI. | 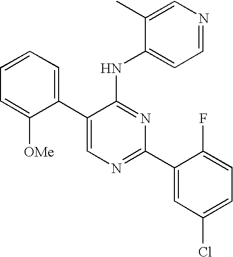 |
| LXVII. | 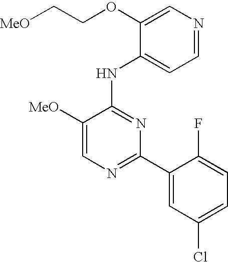 |
| LXVIII. | 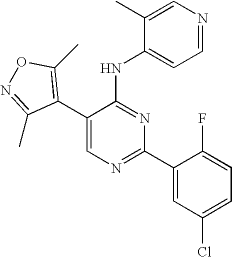 |
| LXIX. | 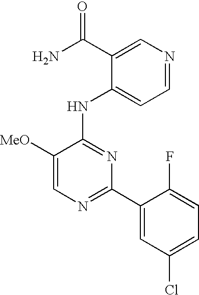 |
| LXX. | 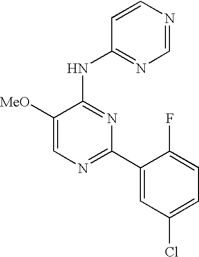 |
| LXXI. | 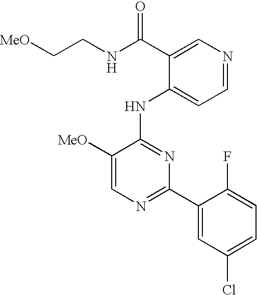 |
| LXXII. | 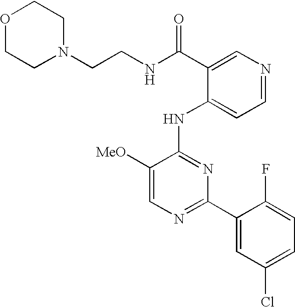 |
| LXXIII. | 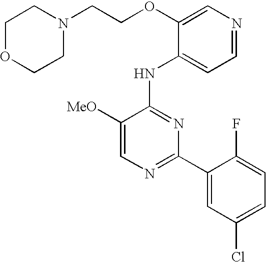 |

Another group of compounds for use in the methods of the present invention is represented by the formula (3)

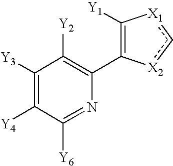

wherein Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₘ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S:

Y₂, Y₃, Y₄, and Ys independently represent hydrogen, alkyl(1-6C), alkoxy(1-6 C), haloalkyl(1-6 C), halo, NH₂, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one o more substituents independently selected from alkyl(1-6 C), alkoxy(a-6 C), haloalkyl(1-6 C), halo, NH₂, NH-alkyl(1-6 C), or NH(CH₂)ₙ—Ph, wherein n is 0-3, and the remainder of Y₂, Y₃, Y₄, and Y₅ represent hydrogen, alkyl(1-6 C), alkoxy(1-6C), haloalkyl(1-6 C), halo, NH2, NH-alkyl(1-6 C), or NH(CH₂)ₙ—Ph wherein n is 0-3; and

one of X₁ and X₂ is N and the other is NR₆, wherein R₆ is hydrogen or alkyl(1-6 C).

As used in formula (3), the double bonds indicated by the dotted lined represent possible tautomeric ring forms of the compounds. Further information about compounds of formula (3) and their preparation is disclosed in WO 02/40468, published May 23, 2002, the entire disclosure of which is hereby expressly incorporated by reference.

Yet another group of compounds for use in the methods of the invention is represented by the following formula (4)

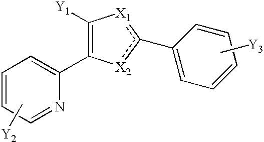

wherein Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)ₙ—Ph, —S—(CH₂)ₙ—Ph, cyano, phenyl, and CO₂R, wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;

Y₂ is H, NH(CH₂)ₙ—Ph or NH-alkyl(1-6 C), wherein n is 0, 1, 2, or 3;

Y₃ is CO₂H, CONH₂, CN, NO₂, alkylthio(1-6 C), —SO₂-alkyl(C₁₋₆), alkoxy(C₁₋₆), SONH₂, CONHOH, NH₂, CHO, CH₂NH₂, or CO₂R, wherein R is hydrogen or alkyl(1-6 C);

one of X₁ and X₂ is N or CR′, and other is NR′ or CHR′ wherein R′ is hydrogen, OH, alkyl(C-16), or cycloalkyl(C₃₋₇); or when one of X₁ and X₂ is N or CR′ then the other may be S or O.

Further details of the compounds of formula (4) and their modes of preparation are disclosed in WO 00/61576 published Oct. 19, 2000, the entire disclosure of which is hereby expressly incorporated by reference.

The compounds of the formulas (1)-(4), may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1)-(4), the compound may also be supplied as a salt with a pharmaceutically acceptable cation.

The compounds of formulas (1)-(4) may also be supplied in the form of a “prodrug” which is designed to release the compound of formulas (1)-(4) when administered to a subject. Prodrug formed designs are well known in the art, and depend on the substituents contained in the compounds of formulas (1)-(4). For example, a substituent containing sulfhydryl could be coupled to a carrier which renders the compound biologically inactive until removed by endogenous enzymes or, for example, by enzymes targeted to a particular receptor or location in the subject.

In the event that any of the substituents in the above formulas contain chiral centers, as some, indeed, do, the compounds include all stereoisomeric forms thereof, both as isolated stereoisomers and mixtures of these stereoisomeric forms.

Synthesis of the Compounds of the Invention

The compounds of formula (1) of the invention may be synthesized from the corresponding 4-halo-2-phenyl quinazoline as described in Reaction Scheme 1; which may be obtained from the corresponding 4-hydroxyquinazoline as shown in Reaction Scheme 2. Alternatively, the compounds can be prepared using anthranylamide as a starting material and benzoylating the amino group followed by cyclization to obtain the intermediate 2-phenyl-4-hydroxy quinazoline as shown in Reaction Scheme 3. Reaction Schemes 4-6 are similar to Reaction Scheme 3 except that an appropriate pyridine or 1,4-pyrimidine nucleus, substituted with a carboxamide residue and an adjacent amino residue, is substituted for the anthranylimide. The compounds of the invention wherein R¹ is H can be further derivatized to comprise other embodiments of R¹ as shown in Reaction Scheme 7.

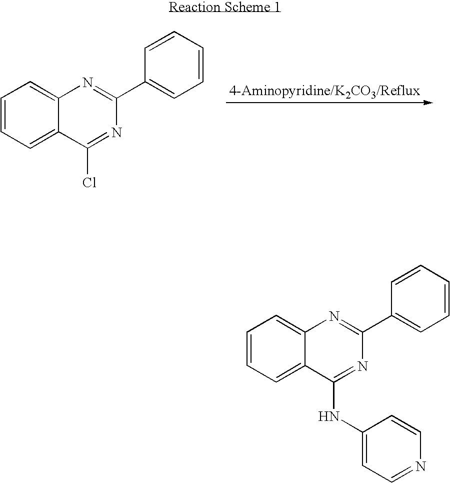

Reaction Scheme 1 is illustrative of the simple conversion of a halogenated quinazoline to compounds of the invention. Of course, the phenyl of the illustration at position 2 may be generalized as R³ and the 4-pyridylamino at position 2 can be generalized to Ar′-L or Ar′-.

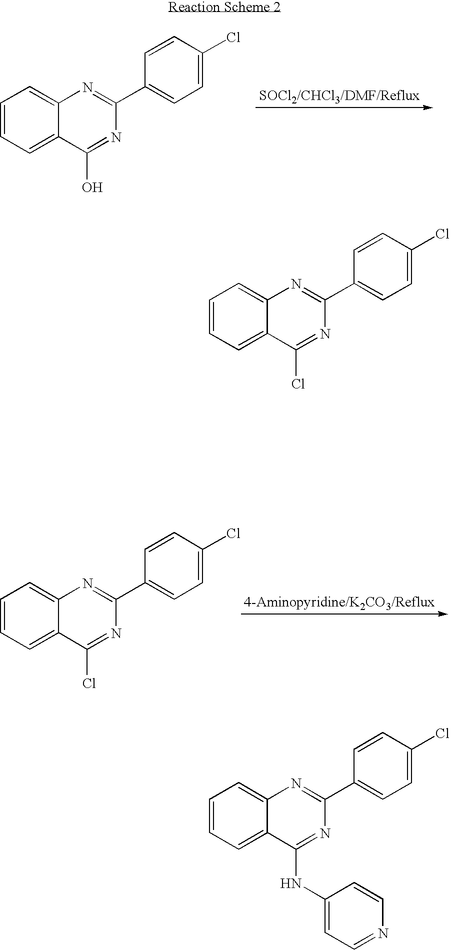

Reaction Scheme 2 can, of course, be generalized in the same manner as set forth for Reaction Scheme 1.

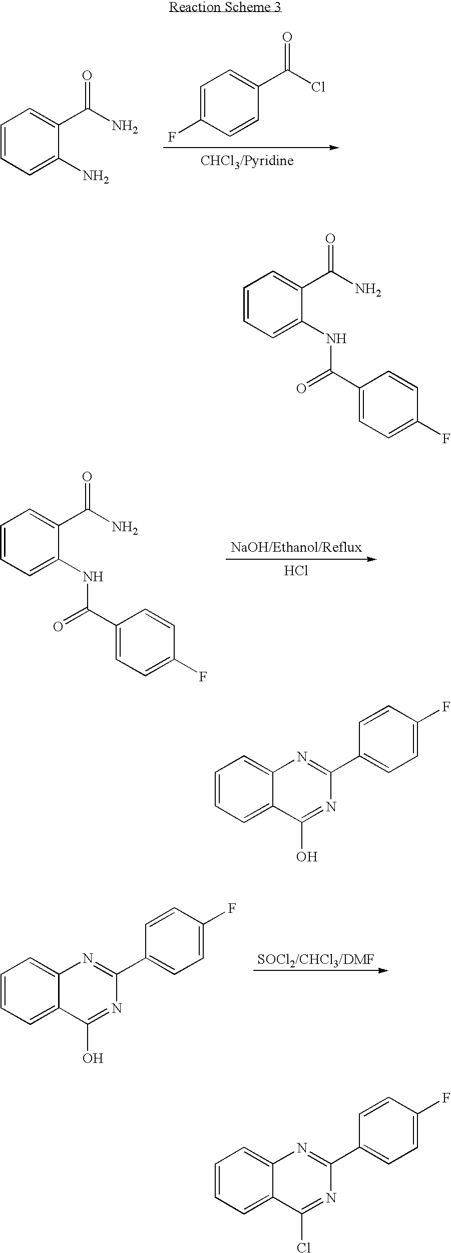

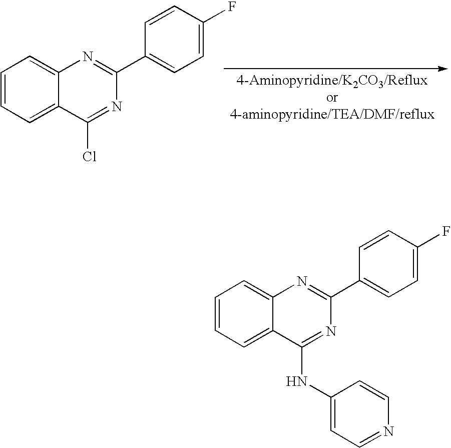

Again, Reaction Scheme 3 can be generalized by substituting the corresponding acyl halide, R³COCl for the parafluorobenzoyl chloride. Further, Ar′ or Ar′-L may be substituted for 4-aminopyridine in the last step.

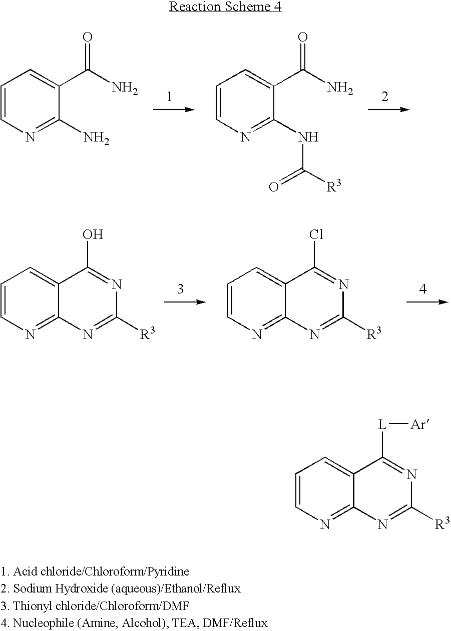

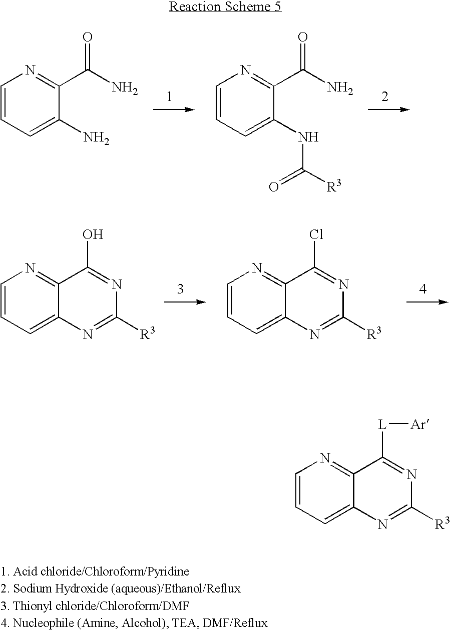

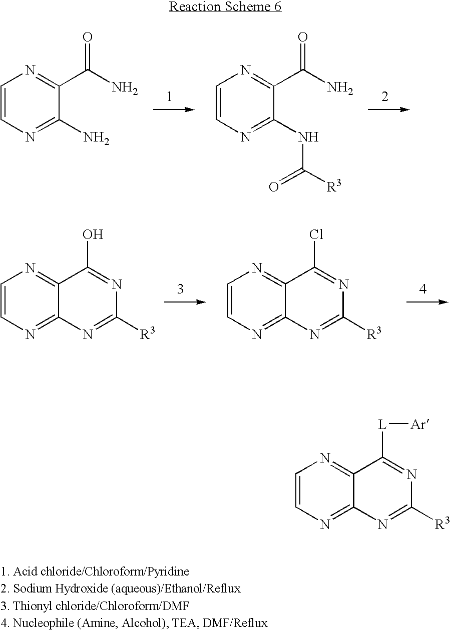

It is seen that Reaction Scheme 1 represents the last step of Reaction Schemes 2-6 and that Reaction Scheme 2 represents the last two steps of Reaction Scheme 3-6.

Reaction Scheme 7 provides conditions wherein compounds of formula (1) are obtained wherein R¹ is other than H.

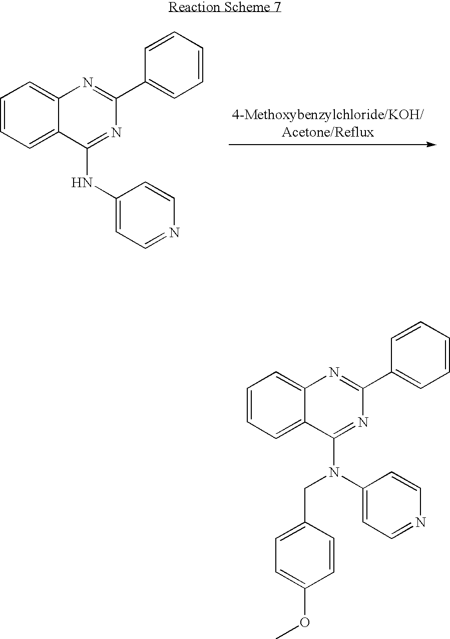

Reaction Scheme 8 is a modification of Reaction Scheme 3 which simply demonstrates that substituents on ring A are carried through the synthesis process. The principles of the behavior of the substituents apply as well to Reactions Schemes 4-6.

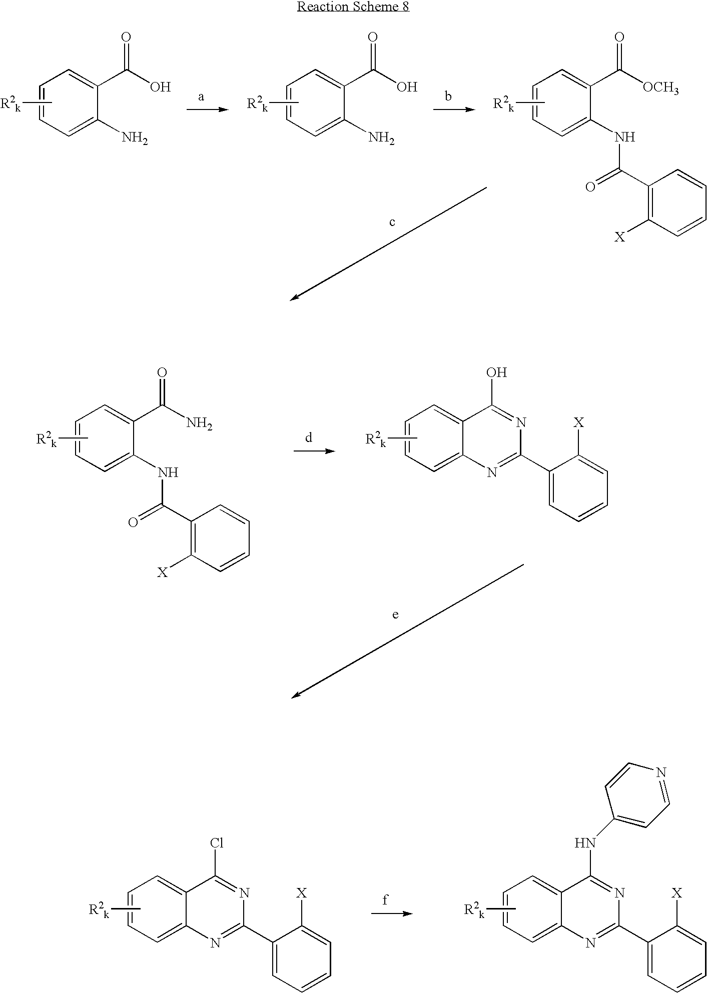

Reaction Scheme 8 shows a modified form of Reaction Scheme 3 which includes substituents R² in the quinazoline ring of formula (1). The substituents are carried throughout the reaction scheme. In step a, the starting material is treated with thionyl chloride in the presence of methanol and refluxed for 12 hours. In step b, the appropriate substituted benzoyl chloride is reacted with the product of step a by treating with the appropriately substituted benzoyl chloride in pyridine for 24 hours. In embodiments wherein X (shown illustratively in the ortho-position) is fluoro, 2-fluorobenzoyl chloride is used as a reagent; where X is (for illustration ortho-chloro), 2-chlorobenzoyl chloride is used.

In step c, the ester is converted to the amide by treating in ammonium hydroxide in an aprotic solvent such as dimethyl formamide (DMF) for 24 hours. The product is then cyclized in step d by treatment with 10 N NaOH in ethanol and refluxed for 3 hours.

The resulting cyclized form is then converted to the chloride in step e by treating with thionyl chloride in chloroform in the presence of a catalytic amount of DMF under reflux for 4 hours. Finally, the illustrated 4-pyridylamino compound is obtained in step f by treating with 4-amino pyridine in the presence of potassium carbonate and DMF and refluxed for 2 hours.

In illustrative embodiments of Reaction Scheme 8, R² may, for example, provide two methoxy substituents so that the starting material is 2-amino-4,5-dimethoxy benzoic acid and the product is, for example, 2-(2-chlorophenyl)-4-(4-pyridylamino)-6,7-dimethoxyquinazoline.

In another illustrative embodiment, R2 provides a single nitro; the starting material is thus, for example, 2-amino-5-nitrobenzoic acid and the resulting compound is, for example, 2-(2-fluorophenyl)-4-(4-pyridylamino)-5-nitroquinazoline.

Reaction Schemes 4-6 can be carried out in a manner similar to that set forth in Reaction Scheme 8, thus carrying along R² substituents through the steps of the process.

In compounds of the invention wherein R² is nitro, the nitro group may be reduced to amino and further derivatized as indicated in Reaction Scheme 9.

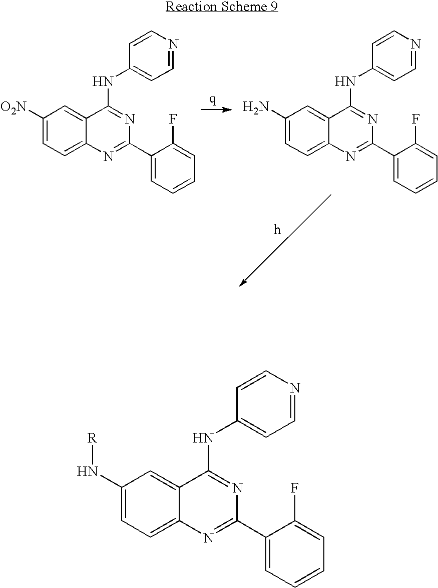

In Reaction Scheme 9, the illustrative product of Reaction Scheme 8 is first reduced in step g by treating with hydrogen and palladium on carbon (10%) in the presence of acetic acid and methanol at atmospheric pressure for 12 hours to obtain the amino compound. The resulting amino compound is either converted to the acyl form (R=acyl) using the appropriate acid chloride in the presence of chloroform and pyridine for four hours, or is converted to the corresponding alkylated amine (R=alkyl) by treating the amine intermediate with the appropriate aldehyde in the presence of ethanol, acetic acid, and sodium triacetoxyborohydride for 4 hours.

While the foregoing exemplary Reaction Schemes are set forth to illustrate the synthetic methods of the invention, it is understood that the substituents shown on the quinazoline ring of the products are generically of the formula (1) as described herein and that the reactants may be substituted accordingly. Variations to accommodate various substituents which represent embodiments of R³ other than the moieties shown in these illustrative examples or as Ar′ in these illustrative examples may also be used. Similarly, embodiments wherein the substituent at position 4 contains an arylalkyl can be used in these schemes. Methods to synthesize the compounds of the invention are, in general, known in the art.

Thus, a number of synthetic routes may be employed to produce the compounds of formula (2). In general, they may be synthesized using reactions known in the art. One useful method, especially with regard to embodiments which contain nitrile substitutions (which also, of course, can be hydrolyzed to the corresponding carboxylic acids or reduced to the amines) is shown in Reaction Scheme 10, shown below. In Reaction Scheme 10, an intermediate wherein the pyrimidine ring is halogenated is obtained; the halide is then displaced by an aryl amine. In this method, the pyrimidine ring is generated in the synthetic scheme, resulting in the compound formed in reactions labeled a.

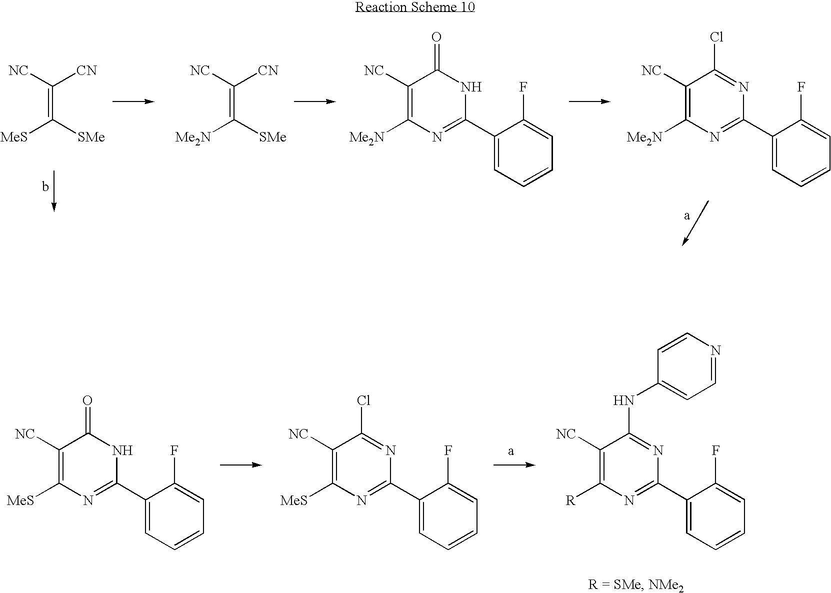

In Reaction Scheme 11, the pyrimidine ring is obtained by cyclizing an amido moiety and, again, a halo group on the pyrimidine ring is displaced by an aryl amide to obtain the compounds of the invention in step b. Further substitution on the resulting invention compound can then also be performed as shown in subsequent steps b¹, b², and b³.

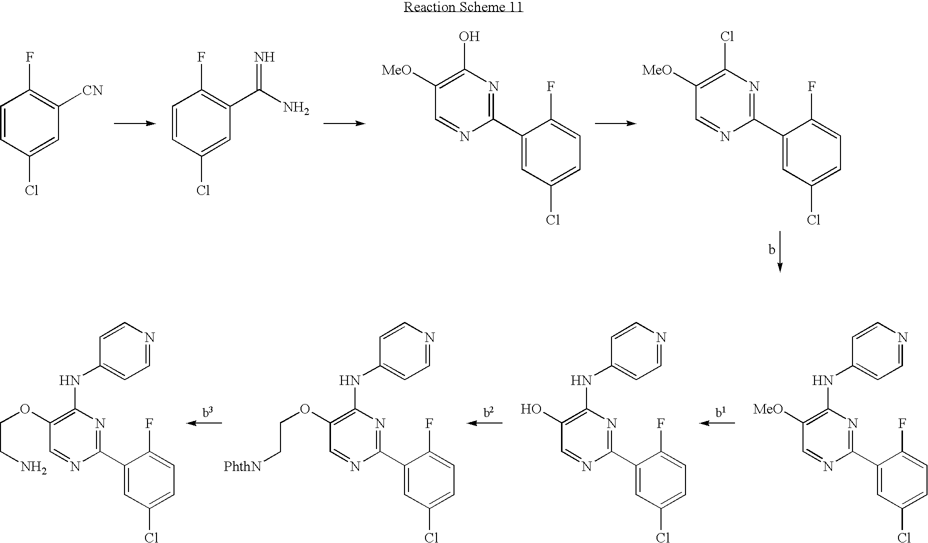

Reaction Schemes 12, 13, 14 and 15, shown below, provide alternative routes to the pyrimidine nucleus, and further substitution thereof.

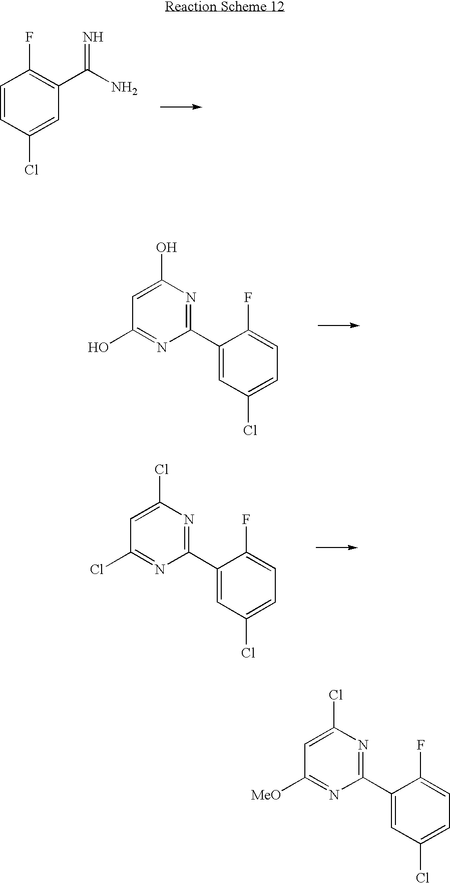

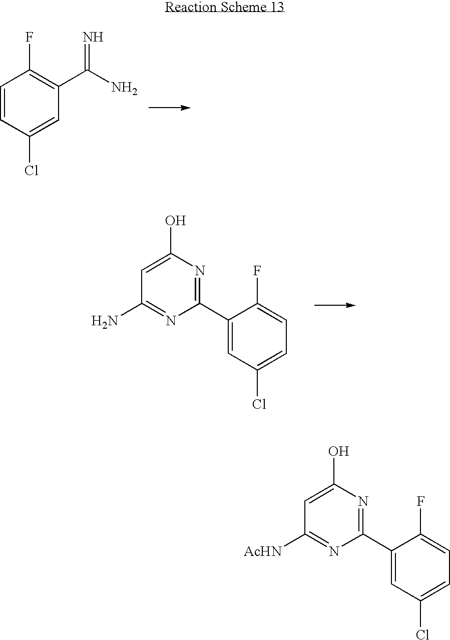

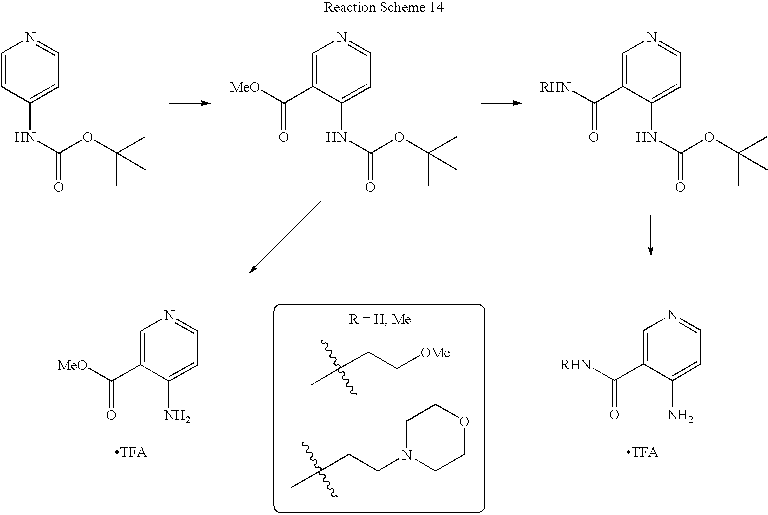

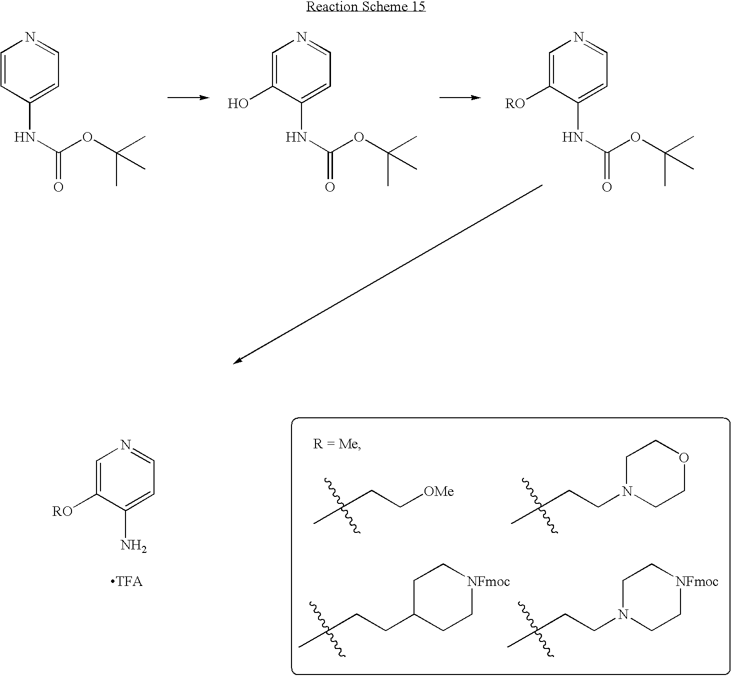

Small organic molecules other than quinazoline derivatives can be synthesized by well known methods of organic chemistry as described in standard textbooks.

Methods of Treatment

There are numerous conditions and diseases that require or may benefit from the improvement of lung function, including, without limitation, emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, and lung injuries resulting from other factors, such as, infectious causes, inhaled toxins, or circulating exogenous toxins, aging and genetic predisposition to impaired lung function.

Chronic bronchitis, emphysema and COPD are typically associated with cigarette smoking, and often coexist, causing abnormalities in lung structure and function, and obstruction of air flow, negatively impacting the quality of life of patients. COPD is commonly used to describe a spectrum of conditions, diseases and symptoms that may occur individually or in combination, including, for example, chronic obstructive bronchitis, emphysema, and chronic airway obstruction. Over the time, as the diseases progress, gradually more serious symptoms can develop. COPD is currently the fourth leading cause of death in the United States.

Current treatments of COPD, and related conditions that require or benefit from the improvement of lung function, include the administration of bronchodilators, such as β-adrenergic agonists, anticholinergic agents, and theophylline, and corticosteroid therapy, although the benefits of these and similar treatments vary from patient to patient, and long term benefits have not been clearly demonstrated.

According to the present inventions, the foregoing diseases and other lung conditions that require or benefit from the improvement of lung function are treated by administration of small molecules specifically binding to the type I TGF-β receptor (TGFβ-R1).

The manner of administration and formulation of the compounds useful in the invention and their related compounds will depend on the nature of the condition, the severity of the condition, the particular subject to be treated, and the judgement of the practitioner; formulation will depend on mode of administration. The small molecule compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis or ophthalmic treatments, or in formulation intended to penetrate the skin or eye. These include lotions, creams, ointments, drops and the like which can be formulated by known methods.

The compounds may also be administered by injection, including intravenous, intramuscular, subcutaneous, intrarticular or intraperitoneal injection. Typical formulations for such use are liquid formulations in isotonic vehicles such as Hank's solution or Ringer's solution.

Alternative formulations include aerosol inhalants, nasal sprays, liposomal formulations, slow-release formulations, and the like, as are known in the art.

A preferred route of administration for the treatment of a disease or condition that requires or benefits from the improvement of lung function is aerosol delivery. Aerosol delivery to various parts of the respiratory tract, including the lungs has been extensively used for delivery of various pharmaceutical agents. Pharmaceutical agents, including small molecule drugs, are generally delivered to the respiratory tract in the form of a fine mist or aerosol which is breathed into the lungs through the nose or mouth of the patient. Typically, a nebulizer is used to convert a liquid into a fine aerosol, and the aerosol is introduced into the lungs by means of a mouthpiece which delivers the aerosol through the mouth only, or by means of a face mask which delivers the aerosol through both the mouth and nose of the patient. The first commercial inhaleable systems developed were developed in the early 1950s, and dispensed drugs for treating asthma or COPD. Various aerosol inhalation devices have been developed and are disclosed, for example, in U.S. Pat. Nos. 4,823,784; 4,106,503; 4,677,975; and 5,479,920. Inhalation devices suitable for the purposes of the present invention specifically include metered dose inhalers (MDIs), nebulisers, and dry powder inhalers (DPIs). A particularly preferred route of administration is intrapulmonary delivery directly to the lungs. The deep lung epithelium, composed of a thin, nonciliated, mucus-free cell layer, offers a very efficient port of entry for the direct delivery of pharmaceuticals, such as small molecule drugs, directly into the patient's blood stream.

The pharmaceutical compositions of the present invention can be prepared by art-known methods, such as those disclosed in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds of formula (1) can be administered as individual active ingredients, or as mixtures of several embodiments of this formula. In addition, the TGF-β inhibitors can be used as single therapeutic agents or in combination with other therapeutic agents. Drugs that could be usefully combined with these compounds include natural or synthetic corticosteroids, particularly prednisone and its derivatives, bronchodilators, monoclonal antibodies targeting cells of the immune system or genes associated with the development or progression of pulmonary diseases, and small molecule inhibitors of cell division, protein synthesis, or mRNA transcription or translation, or inhibitors of immune cell differentiation or activation.

As implicated above, although the compounds of the invention may be used in humans, they are also available for veterinary use in treating non-human mammalian subjects.

Animal Models

Prior to administration to human or veterinary patients, the safety and efficacy of small molecule drug candidates is typically tested in in vitro and in vivo assays, including animal models of the target disease.

In view of the similarities in lung development and lung structure between humans and other mammals, animal models can provide valuable insights into the pathogenesis of diseases and conditions characterized by reduced or compromised lung function, and may be developed for testing drug candidates. In particular, the mice have been considered as preferred for developing animal models because the mouse genome has been extensively studied and sequences, and close similarities exist with the human genome. Since many of the lung conditions benefiting from the improvement of lung function are associated with smoking, and have complex etiologies that are not clearly understood, traditionally meaningful animal models have been scarce. However, in recent times several groups have made significant progress to remedy this situation.

Animal models of COPD have been discussed at the First International Conference on Animal Models of Chronic Obstructive Pulmonary Disease, Certosa di Pontignano, University of Siena, Italy, September 30-Oct. 2, 2001. A meeting report authored by David Hele has been published in Respir Res 3:12 (2002).

Emphysema-induced changes in lung function can be demonstrated in the rat, using elastase to generate an emphysematous pathology.

Smoking models have been developed by several laboratories. Cigarette smoke-induced lesions in animal models have been shown to be similar to those observed in humans. Mice, such as B6C3F1 mice, were demonstrated to show an inflammatory and emphysematous response to chronic exposure to cigarette smoke. After long term exposure to cigarette smoke, A/J mice showed a faster development of emphysema than C57BI/6J mice used for comparison. Other researchers have suggested that it is important to check the antiprotease and antioxidant status of an animal strain before establishing an animal model of COPD. It has been shown that C57BI/6J and DBA/2J mice (reduced antielastase and increased sensitivity to antioxidants) were more responsive to cigarette smoke exposure than were ICR mice, which have a normal level of antielastase and lack sensitivity to antioxidants.

In non-cigarette smoke driven models ozone, lipopylsaccharides, sulphur dioxide, nitrogen dioxide, diesel particles, and the like have been used to produce aspects of COPD, such as cough, inflammation, and mucus hypersecretion.

Transgenic animal, e.g. mouse models are also known in the art. For example, the development of spontaneous emphysema has been described in the pallid mouse, an animal that has reduced elastase inhibitory capacity. Emphysema development can be accelerated by treatment with formyl-methionyl-leucyl phenylalanine or exposure to cigarette smoke.

For further discussion of animal models of COPD see, also Dawkins and Stockley, Thorax 56:973-977 (2001).

Further details of the invention will be apparent from the following non-limiting examples.

### EXAMPLE 1

Effect of a Representative Compound of Formula (1) on Respiratory Rate, Tidal Volume and Total BALF IL-6 in a 5-Day Bleomycin-Induced Lung Injury Model Material and Methods

Animal Model

Male Sprague-Dawley rats weighing 225 to 250 were purchased from Charles River Laboratories, Inc. Rats were housed in groups of two in an animal facility provided with filtered air and constant temperature and humidity. All animal maintenance was in accordance with Scios' guidelines for animal welfare. The rats were allowed to acclimate to the new environment for one week before treatment. A 12:12 hour light-dark cycle was maintained, and the animals had free access to ad libitum food and water.

Protocol

| | | | Rx2 (1 Day after |
|---|---|---|---|
| Group | n | Rx1 | Rx 1) |
| 1 | 24 | Saline | 1% MC |
| 2 | 24 | Bleomycin | 1% MC |
| 3 | 6 | Bleomycin | 10 mg/kg |
| | | | Compound A |
| 4 | 12 | Bleomycin | 30 mg/kg |
| | | | Compound A |
| 5 | 24 | Bleomycin | 60 mg/kg |
| | | | Compound A |

Treatment Protocol

Day 0: To induce pulmonary injury, rats were intubated with 0.5 ml of saline or 0.5 ml of 2.0 unit/ml of bleomycin by intratracheal injection under anesthesia. The anesthetic solution used was a mixture of 0.4 ml of ketamine (100 mg/ml) and 0.25 ml of xylazine (20 mg/ml) at a dose of 1.3 ml/kg.

Day 1 to Day 5:

Group 1 & 2: Rats were weighed and orally dosed with 5 ml/kg of 1% methyl cellulose (MC) two times a day.

Group 3: Rats were weighed and orally dosed with 5 ml/kg of 2.0 mg/ml of Compound A two times a day.

Group 4: Rats were weighed and orally dosed with 5 ml/kg of 6.0 mg/ml of Compound A two times a day.

Group 5: Rat were weighed and orally dosed injected with 5 ml/kg of 12.0 mg/ml of Compound A two times a day.

Day 4: After dosing, rats were placed in the Buxco system to measure lung functions.

Day 5: After dosing, rats were sacrificed, BALF was collected and stored at −80° C. for IL-6 analysis.

Lung Functions

To measure other lung functions, the Buxco whole body plethysmograph system was used (Buxco Electronics, Inc., Sharon, Conn.), to measure respiratory rate, and tidal volume. Briefly, the Buxco system was first calibrated, then rats were placed into the whole body unrestrained plethysmographs for 1 hour to be acclimatized, and then lung functions were continuously collected for 30 minutes by the BioSystem XA for Windows Software.

Bronchoalveolar Lavage Fluid (BALF) Collections

Rats were sacrificed by overdose of ketamine/xylazine cocktail, and then trachea, heart and lung were removed en bloc. BALF was collected from the lungs slowing injecting 4 ml of 1× PBS into the lungs and slowly withdrawing the 1× PBS out of the lungs. This process is repeated for three times. BALF was then centrifuged at 4° C. for 15 minutes at 3000 rpm. The supernatant was saved and stored at −80° C. for measurement of IL-6, and protein.

Determination of IL-6

Total BALF IL-6 was measured by the R & D System ELISA Kit (cat #: R6000).

Statistical Analysis

The data were analyzed using a one-way analysis of variance (ANOVA) with a Bonferroni's multiple comparisons post tests. A value of p≦0.05 was considered statistically significant. Values are reported as mean±SD.

Results:

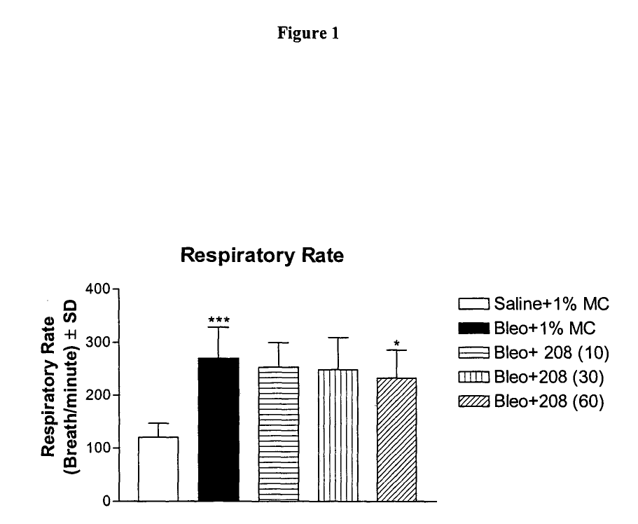

The results are illustrated on FIGS. 1-3.

FIG. 1 shows the respiratory rate measured in control (MC-treated) and bleomycin-treated animals as well as animals treated with 10 mg/kg, 30 mg/kg, and 60 mg/kg doses of Compound A following bleomycin treatment as described above. In FIG. 1 *** indicates p<0.001, and * indicates p<0.05. The first and second graphs show that bleomycin significantly increases the respiratory rate in rats (Saline+1% MC versus Bleo+1% MC) relative to saline-treated control animals. The Figure further shows that Compound A significantly reduces respiratory rate induced by bleomycin (Bleo+1% MC versus Bleo-Compound A).

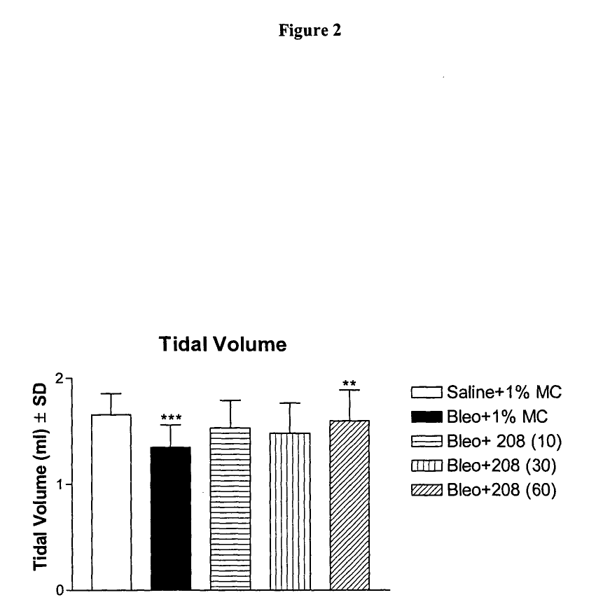

FIG. 2 shows the tidal volume measured in control (MC-treated) and bleomycin-treated animals as well as animals treated with 10 mg/kg, 30 mg/kg, and 60 mg/kg doses of Compound A following bleomycin treatment as described above. In FIG. 2 *** indicates p<0.001, and * indicates p<0.01. The first and second graphs show that bleomycin significantly decreases tidal volume in rats (Saline+1% MC versus Bleo+1% MC) compared to saline-treated control. The Figure further shows that treatment with Compound A significantly increases tidal volume induced by bleomycin (Bleo+1% MC versus Bleo-Compound A).

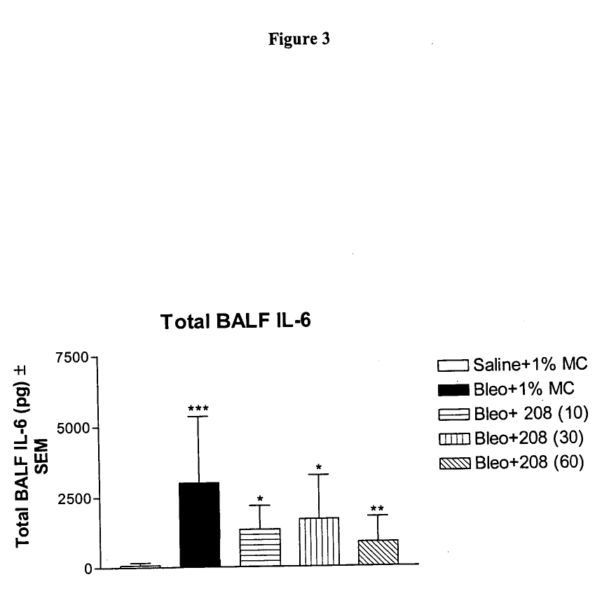

FIG. 3 shows the effect of Compound A on total BALF IL-6 induced by bleomycin. In FIG. 3 * indicates p<0.05; ** indicates p<0.01; and ***indicates p<0.001. The first and second graphs show that bleomycin significantly increases total BALF IL-6 in rats (Saline+1% MC versus Bleo+1% MC). The Figure further shows that treatment with Compound A significantly decreases total BALF IL-6 induced by bleomycin (Bleo+1% MC versus Bleo-Compound A (10), bleo+1% MC versus Bleo-Compound A (30), bleo+1% MC versus Bleo-Compound A (60)).

Conclusion:

Bleomycin-treated rats that dosed with Compound A show a significant improvement in lung functions, and a significant decrease in total BALF IL-6 compared to bleomycin-treated rats orally dosed with the 1% MC. Since these data were obtained in a 5-day bleomycin study, they are indicative of the ability of Compound A to improve lung function following acute lung injury, before the development of fibrosis.

### EXAMPLE 2

Effect of a Representative Compound of Formula (1) on Total Lung Capacity and Lung Permeability in a 5-Day Bleomycin-Induced Lung Injury Model Material and Methods

Animal Model

Male Sprague-Dawley rats weighing 225 to 250 were purchased from Charles River Laboratories, Inc. Rats were housed in groups of two in the animal facility provided with filtered air and constant temperature and humidity. All animal maintenance was in accordance with Scios' guidelines for animal welfare. The rats were allowed to acclimate to the new environment for one week before all treatment. A 12:12 hour light-dark cycle was maintained, and the animals had free access to ad libitum food and water.

Protocol

| | | | Rx2 (1 Day after |
|---|---|---|---|
| Group | n | Rx1 | Rx 1) |
| 1 | 4 | Saline | 1% MC |
| 2 | 4 | Bleomycin | 1% MC |
| 3 | 4 | Bleomycin | 60 mg/kg |
| | | | Compound A |

Treatment Protocol

Day 0: To induce pulmonary injury, rats were intubated with 0.5 ml of saline or 0.5 ml of 2.0 unit/ml of bleomycin by intratracheal injection under anesthesia. The anesthetic solution used is a mixture of 0.4 ml of ketamine (100 mg/ml) and 0.25 ml of xylazine (20 mg/ml) at a dose of 1.3 ml/kg.

Day 1 to Day 5:

Group 1 & 2: Rats were weighed and orally dosed with 5 ml/kg of 1% methyl cellulose (MC) two times a day.

Group 3: Rats were weighed and orally dosed with 5 ml/kg of 12.0 mg/ml of Compound A two times a day.

Day 5: After dosing, rats were injected intravenously with 3 ml/kg of 10 mg/ml of rhodamine labeled dextran. Two hours after injection of rhodamine labeled dextran, rats were sacrificed, and lungs were inflated and fixed for histological analysis

Lung Functions

To estimate total lung capacity, lungs were inflated with 4% formalin at a constant pressure of 15 cm of water. Total lung capacity is equal to the volume of 4% formalin used to inflate the lung. The maximum volume to inflate the lung is 10 ml.

Histology

Lungs were first inflated with 4% formalin at a constant pressure of 15 cm of water and the maximum volume to be inflated is 10 ml. After inflation, the inflated lungs were then fixed in 10% formalin for 48 hours. Each lung was cut into seven segments and each segment was embedded in O.C.T. Two six micrometer frozen sections were cut from each segment. One for H & E stain and one unstained for rhodamine labeled dextran analysis.

Tissues analyses were totally blinded and randomized using the NikonE600 microscope equipped with spot digital camera aided by Image-Pro-Plus 4.5 software. To examine the vascular permeability in the alveolar wall, tissues were analyzed for the presence of the positive rhodamine-β-isothiocyanate (RITC)-labeled dextran under the NikonE600 fluorescence microscope using rhodamine filter at magnification of 600×. Ten fields from each seven sections (70 fields per lung) were evenly chosen and the positive fluorescent signals were measured by Image-Pro-Plus-Mirco.

Statistical Analysis

The data were analyzed using a one-way analysis of variance (ANOVA) with a Bonferroni's multiple comparisons post tests. A value of p≦0.05 was considered statistically significant. Values are reported as mean±SD.

Results:

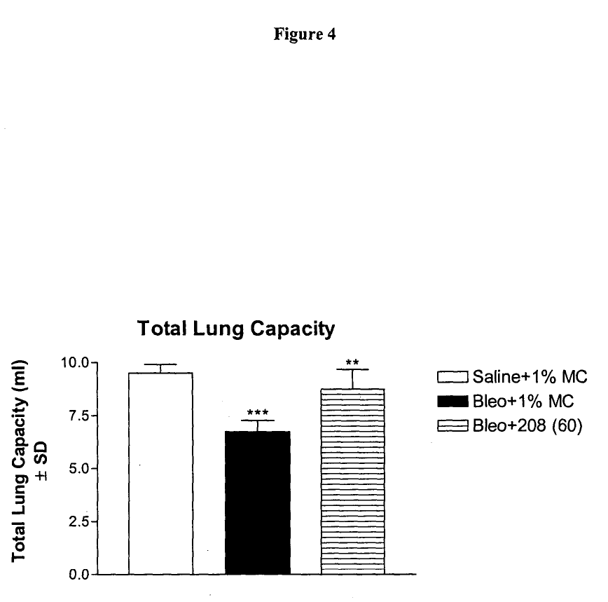

The effect of Compound A on total lung capacity following bleomycin-induced lung injury is shown in FIG. 4. In FIG. 4, ** indicates p<0.01; and *** indicates p<0.001. The first two graphs show that bleomycin significantly decreases total lung capacity in rats (Saline+1% MC versus Bleo+1% MC). The third graph shows that treatment with Compound A as described above significantly increases total lung capacity induced by bleomycin (Bleo+1% MC versus Bleo-Compound A (60)).

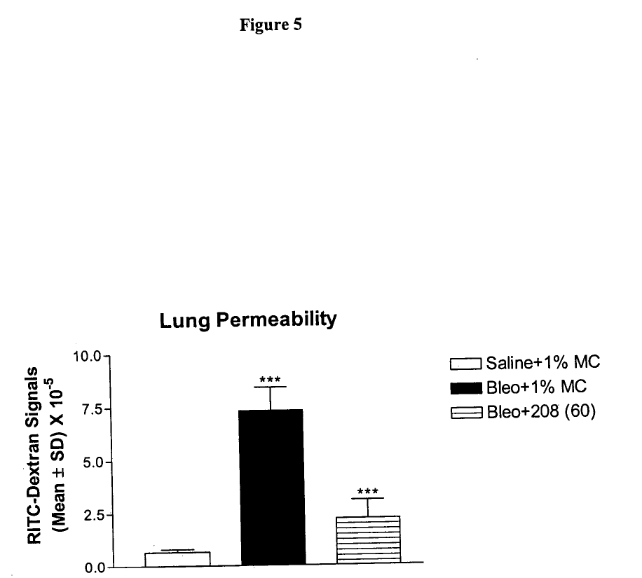

The effect of Compound A on lung permeability following bleomycin-induced lung injury is shown in FIG. 5. In FIG. 5, *** represents p<0.0001. The first two graphs show that bleomycin significantly increases lung permeability in rats (Saline+1% MC versus Bleo+1% MC). The third graph shows that treatment with Compound A as described above significantly decreases lung permeability induced by bleomycin (Bleo+1% MC versus Bleo-Compound A (60)).

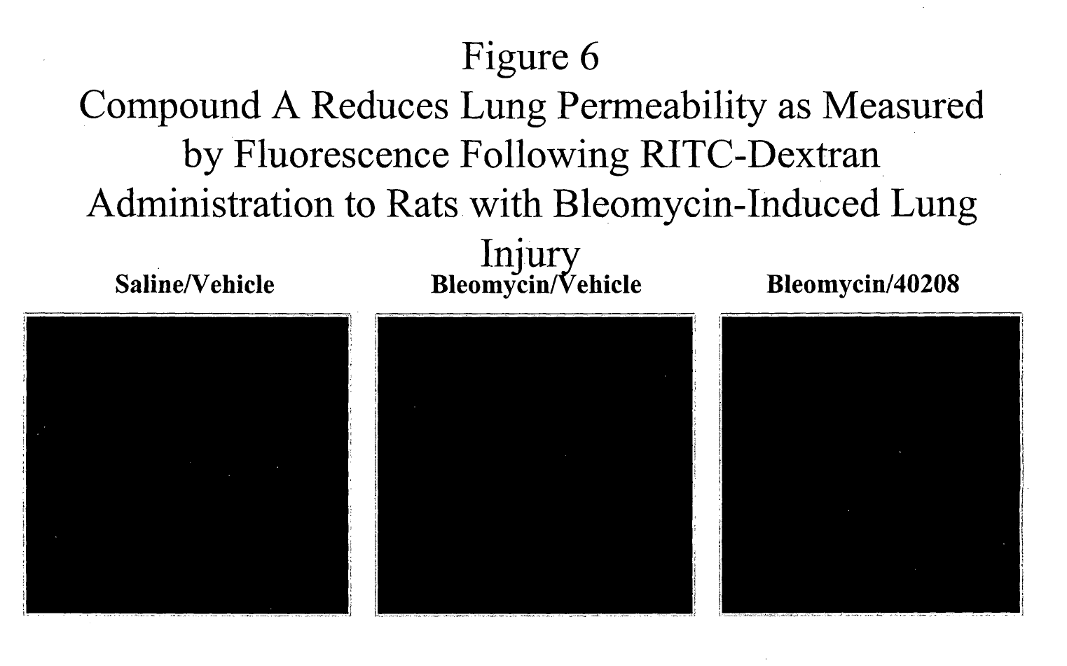

FIG. 6 shows the effect of Compound A on lung permeability following bleomycin-induced lung injury, as measured by fluorescence following RITC-dextran administration to rats as described above.

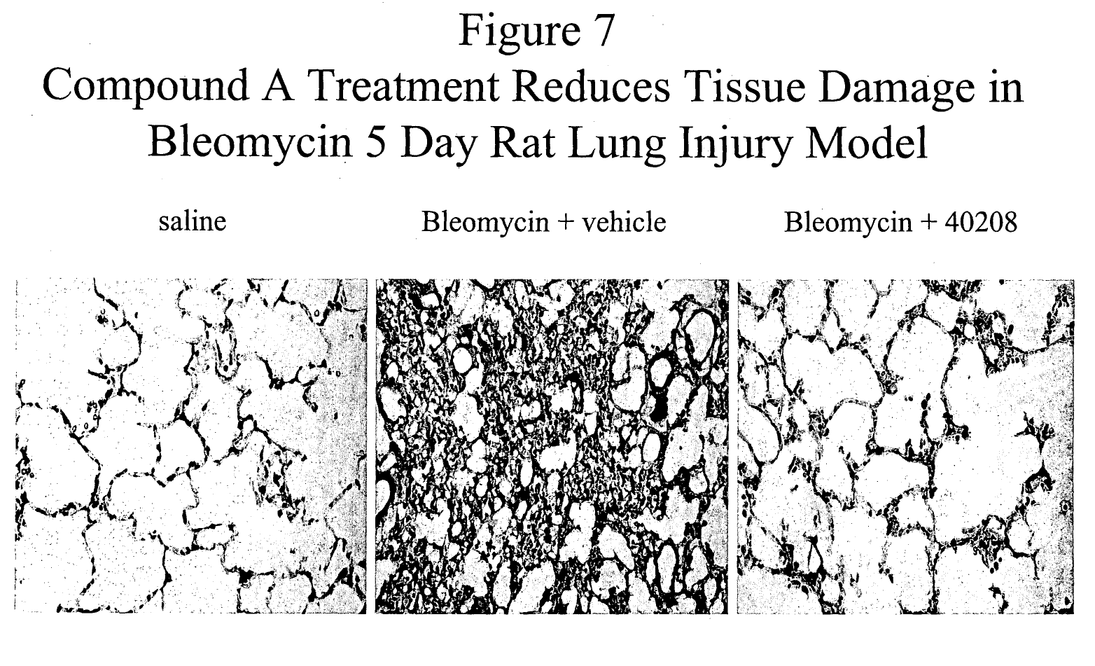

FIG. 7 shows H & E stained tissue sections after bleomycin treatment and subsequent treatment with Compound A. The tissue sections clearly show that treatment with Compound A reduces tissue damage in bleomycin 5-day rat lung injury model.

Conclusion:

Bleomycin-treated rats orally dosed with Compound A show a significant improvement in lung function, and a significant decrease in lung permeability compared to bleomycin-treated rats orally dosed with the 1% MC. Since these data were obtained in a 5-day bleomycin study, they are indicative of the ability of compound A to improve lung function following acute lung injury, before the development of fibrosis.

### EXAMPLE 3

Effect of a Representative Compound of Formula (1) on Lung Hyroxyproline Content Following Bleomycin-Induced Lung Fibrosis Material and Methods

Animal Model

Male Sprague-Dawley rats weighing 225 to 250 were purchased from Charles River Laboratories, Inc. Rats were housed in groups of two in the animal facility provided with filtered air and constant temperature and humidity. All animal maintenance was in accordance with Scios' guidelines for animal welfare. The rats were allowed to acclimate to the new environment for one week before all treatment. A 12:12 hour light-dark cycle was maintained, and the animals had free access to ad libitum food and water.

Protocol

| | | | Rx2 (1 Day after |
|---|---|---|---|
| Group | n | Rx1 | Rx 1) |
| 1 | 6 | No Rx | No Rx |
| 2 | 8 | Saline | Saline |
| 3 | 8 | Bleomycin | Saline |
| 4 | 7 | Bleomycin | 30 mg/kg |
| | | | Compound B |
| 5 | 10 | Bleomycin | 8 mg/kg |
| | | | Triamcinolone |

Treatment Protocol

Day 0: To induce pulmonary fibrosis, rats were intubated with 0.5 ml of saline or 0.5 ml of 1.0 unit/ml of bleomycin by intratracheal injection under anesthesia. The anesthetic solution used is a mixture of 0.4 ml of ketamine (100 mg/ml) and 0.25 ml of xylazine (20 mg/ml) at a dose of 1.3 ml/kg.

Day 1 to Day 14:

Group 1: Rats were weighed daily

Group 2 & 3: Rats were weighed and orally dosed with 5 ml/kg of saline three times a day

Group 4: Rats were weighed and orally dosed with 5 ml/kg of 6.0 mg/ml of Compound B three times a day

Group 5: Rats were weighed and intraperitoneally injected with 1 ml/kg of 8 mg/ml of Triamcinolone every other day

Day 14: After dosing, rats were sacrificed by overdose of the ketamine/xylazine cocktail, and then trachea, heart and lung were removed en bloc. All lung lobes were dissected and collected and stored in -80° C. for hydroxyproline assays.

Determination of Hydroxyproline

To estimate the total amount of collagen in fibrotic lungs, the hydroxyproline content of the whole lung was measured in each group according to the method described by Woessner Biochim Biophys Acta. 1967 Jun. 27;140(2):329-38.

Briefly, lungs were harvested and homogenized in 15 ml of 1×PBS with a Polytron homogenizer. Each sample (1 ml) was digested in 2 ml of 6 N HCl for 18 hours at 110C. The samples were neutralized with 3 N NaOH. The hydroxyproline content was the measured using the method of Woessner.

Statistical Analysis

The data were analyzed using a one-way analysis of variance (ANOVA) with a Bonferroni's multiple comparisons post tests. A value of p≦0.05 was considered statistically significant. Values are reported as mean±SD.

Results:

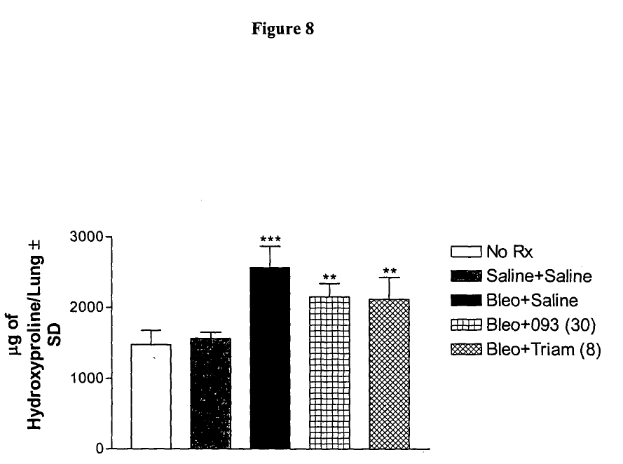

FIG. 8 shows that both Triamcinolone and Compound B attenuated bleomycin-induced lung fibrosis in rats by significantly reducing lung hydroxyproline content. In FIG. 8, ** represents p<0.01, and *** represents p<0.001. The first three graphs demonstrate that bleomycin significantly increased the amount of hydroxyproline in rats (Saline+Saline versus Bleo+Saline). The third and fourth graphs show that both Triamcinolone and Compound B attenuated the effect of bleomycin on the amount of hydroxyproline as the amount of hydroxyproline in the bleo+093 and Bleo+Triam groups were significantly less than bleo+saline.

Conclusion:

Compound B attenuated bleomycin-induced lung fibrosis in rats by significantly reducing lung hydroxyproline content.

### EXAMPLE 4

Effect of a Representative Compound of Formula (1) on Total Lung Capacity and Lung Fibrosis in a 14-Day Bleomycin-Induced Lung Injury Model Material and Methods

Animal Model

Male Sprague-Dawley rats weighing 225 to 250 were purchased from Charles River Laboratories, Inc. Rats were housed in groups of two in the animal facility provided with filtered air and constant temperature and humidity. All animal maintenance was in accordance with Scios' guidelines for animal welfare. The rats were allowed to acclimate to the new environment for one week before all treatment. A 12:12 hour light-dark cycle was maintained, and the animals had free access to ad libitum food and water.

Protocol

| | | | Rx2 (1 Day after |
|---|---|---|---|
| Group | n | Rx1 | Rx 1) |
| 1 | 4 | Saline | 1% MC |
| 2 | 4 | Bleomycin | 1% MC |
| 3 | 3 | Bleomycin | 60 mg/kg |
| | | | Compound A |

Treatment Protocol

Day 0: To induce pulmonary fibrosis, rats were intubated with 0.5 ml of saline or 0.5 ml of 2.0 unit/ml of bleomycin by intratracheal injection under anesthesia. The anesthetic solution used is a mixture of 0.4 ml of ketamine (100 mg/ml) and 0.25 ml of xylazine (20 mg/ml) at a dose of 1.3 ml/kg.

Day 1 to Day 14:

Group 1 & 2: Rats were weighed and orally dosed with 5 ml/kg of 1% methyl cellulose (MC) two times a day.

Group 3: Rats were weighed and orally dosed with 5 ml/kg of 12.0 mg/ml of Compound A two times a day.

Day 14: After dosing, rats were sacrificed by overdose of the ketamine/xylazine cocktail, and lungs were inflated and fixed for histological analysis

Lung Functions

To estimate total lung capacity, lungs were inflated with 4% formalin at a constant pressure of 15 cm of water. Total lung capacity is equal to the volume of 4% formalin used to inflate the lung. The maximum volume to inflate the lung is 10 ml.

Histology

Lungs were first inflated with 4% formalin at a constant pressure of 15 cm of water and then fixed in 10% formalin for 48 hours. Each lung was cut into seven segments and each segment was embedded in O.C.T. Six micrometer sections were cut from each segment. The slides were stained for H & E and trichrome for imaging analysis.

Imaging analysis was totally blinded and randomized using the NikonE600 microscope equipped with spot digital camera aided by Image-Pro-Plus 4.5 software.

Statistical Analysis

The data were analyzed using a one-way analysis of variance (ANOVA) with a Bonferroni's multiple comparisons post tests. A value of p<0.05 was considered statistically significant. Values are reported as mean±SD.

Results:

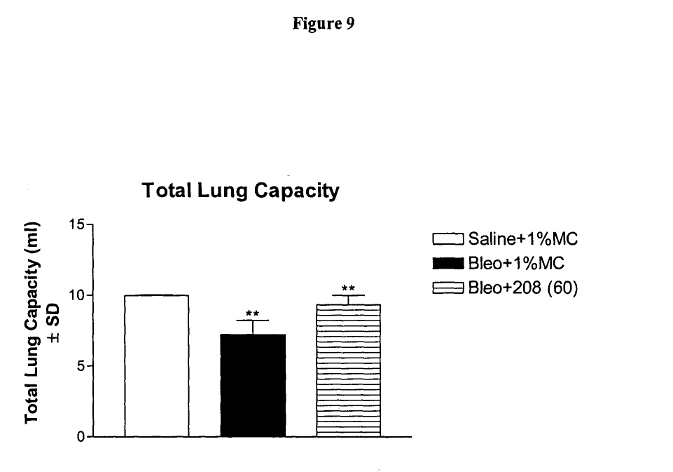

FIG. 9 shows the effect of Compound A on total lung capacity following bleomycin-induced lung fibrosis. In the Figure, ** represents p<0.01. As shown in FIG. 8, bleomycin significantly decreases total lung capacity in rats (Saline+1% MC versus Bleo+1% MC), and Compound A significantly increases total lung capacity induced by bleomycin (Bleo+1% MC versus Bleo-Compound A (60)).

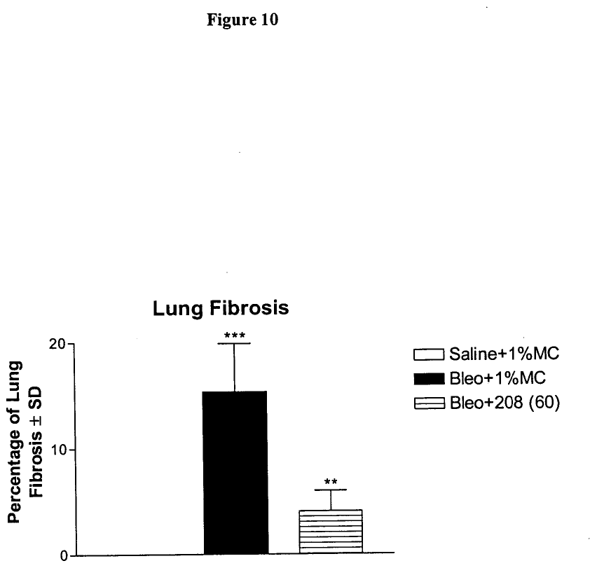

FIG. 10 shows that bleomycin induces lung fibrosis in rats (Saline+1% MC versus Bleo+1% MC), and Compound A significantly reduces lung fibrosis induced by bleomycin (Bleo+1% MC versus Bleo-Compound A (60)).

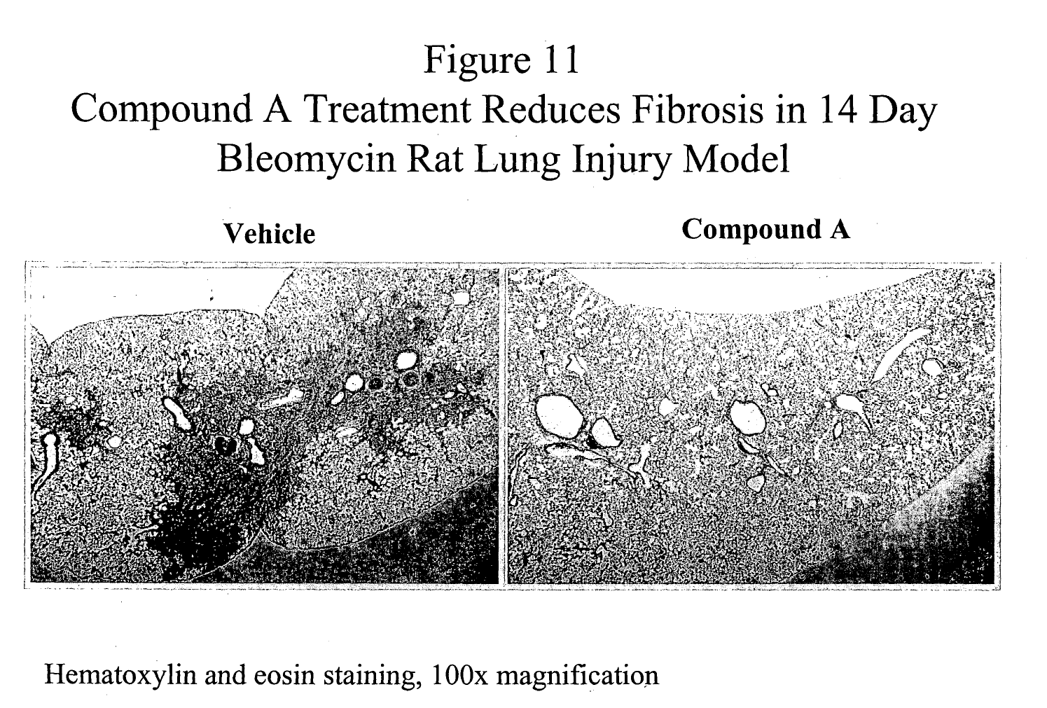

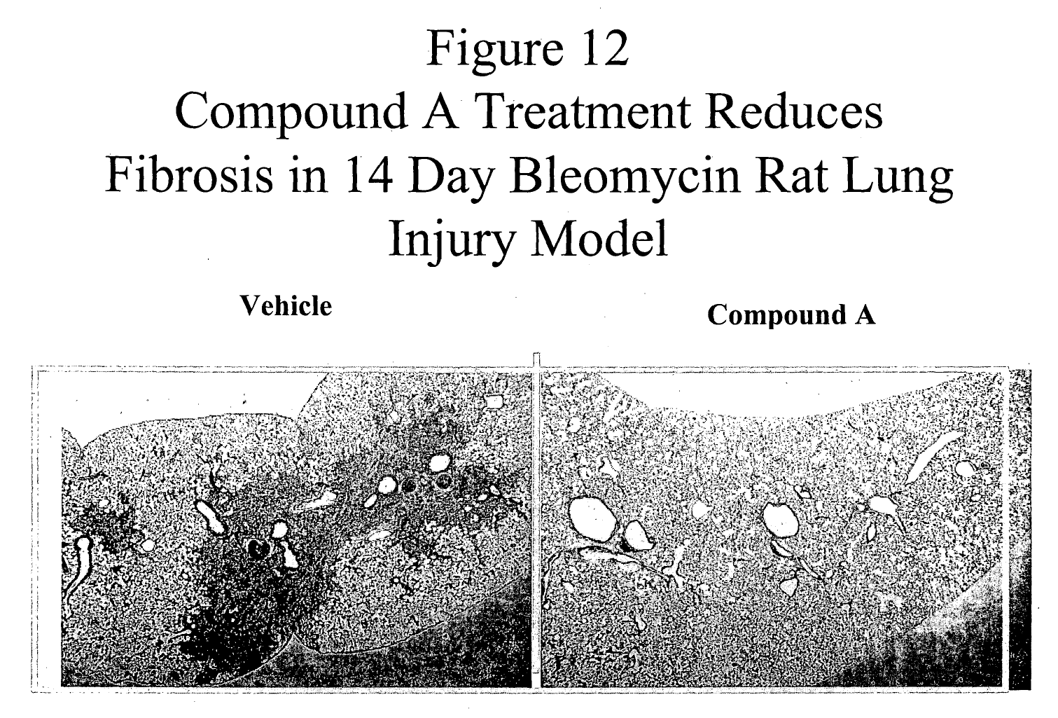

FIGS. 11 and 12 are histology pictures showing that treatment with Compound A reduces fibrosis in this 14-day bleomycin rat lung injury model.

### EXAMPLE 5

Identifying Compounds for Use in the Methods of the Invention

Compounds that are useful for the invention can be tested for their ability to inhibit TGF-β by a TGFβ-R1 autophosphorylation protocol. This was conducted as follows: Compound dilutions and reagents were prepared fresh daily. Compounds were diluted from DMSO stock solutions to 2 times the desired assay concentration, keeping final DMSO concentration in the assay less than or equal to 1%. TGFβ-R1 was diluted to 4 times the desired assay concentration in buffer+DTT. ATP was diluted into 4× reaction buffer, and gamma-33P-ATP was added at 60uCi/mL.

The assay was performed by adding 10 μl of the enzyme to 20 μl of the compound solution. The reaction was initiated by the addition of 10 μl of ATP mix. Final assay conditions included 10 uM ATP, 170 nM TGF R1, and 1M DTT in 20 mM MOPS, pH7. The reactions were incubated at room temperature for 20 minutes. The reactions were stopped by transferring 23 μl of reaction mixture onto a phosphocellulose 96-well filter plate, which had been pre-wetted with 15 ul of 0.25M H3PO4 per well. After 5 minutes, the wells were washed 4× with 75 mM H3PO4 and once with 95% ethanol. The plate was dried, scintillation cocktail was added to each well, and the wells were counted in a Packard TopCount microplate scintillation counter.

All references cited throughout the specification are expressly incorporated herein by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, and the like. All such modifications are within the scope of the claims appended hereto.

What is claimed is:

1. A method for the improvement of lung function, comprising administering to a mammalian subject diagnosed with a disease or condition benefiting from the improvement of lung function an effective amount of a molecule capable of inhibiting a biological activity mediated by a TGFβ-R1 kinase receptor.

2. The method of claim 1 wherein said disease or condition benefiting from the improvement of lung function is selected from the group consisting of emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, lung injuries resulting from infectious causes, inhaled toxins, or circulating exogenous toxins, aging and genetic predisposition to impaired lung function.

3. The method of claim 1 wherein said disease or condition benefiting from the improvement of lung function involves acute lung injury.

4. The method of claim 1 wherein said disease or condition benefiting from the improvement of lung function is unaccompanied by lung fibrosis.

5. The method of claim 1 wherein said disease or condition benefiting from the improvement of lung function is at a stage when lung fibrosis is not a major symptom.

6. The method of claim 1 wherein said molecule specifically binds to said TGFβ-R1 kinase receptor.

7. The method of claim 1 wherein said molecule additionally inhibits a biological activity mediated by p38 kinase.

8. The method of claim 1 wherein said molecule preferentially inhibits a biological activity mediated by TGF-β-R1 kinase relative to a biological activity mediated by p38 kinase.

9. The method of claim 1 wherein said compound is a non-peptide small molecule.

10. The method of claim 9 wherein said compound is a small organic molecule.

11. The method of claim 10 wherein said small organic molecule is other than an imidazole derivative.

12. The method of claim 10 wherein said molecule is a compound of formula (1) 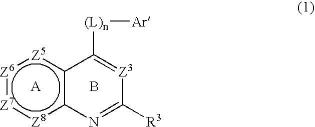
or the pharmaceutically acceptable salts thereof
wherein R³ is a noninterfering substituent;
each Z is CR2 or N, wherein no more than two Z positions in ring A are N, and
wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

13. The method of claim 12 wherein said compound is a quinazoline derivative.

14. The method of claim 13 wherein Z³ is N; and Z¹-Z⁸ are CR².

15. The method of claim 13 wherein Z³ is N; and at least one of Z¹-Z⁸ is nitrogen.

16. The method of claim 13 wherein R³ is an optionally substituted phenyl moiety.

17. The method of claim 16 wherein R³ is selected from the group consisting of 2-, 4-, 5-, 2,4- and 2,5-substituted phenyl moieties.

18. The method of claim 17 wherein at least one substituent of said phenyl moiety is an alkyl(1-6C), or halo.

19. The method of claim 10 wherein said small organic molecule is a compound of formula (2) 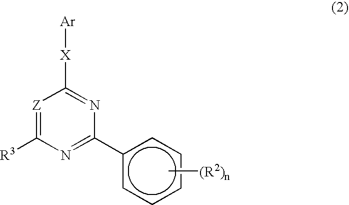
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5.

20. The method of claim 10 wherein said small organic molecule is a compound of formula (3) 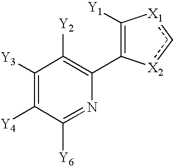
wherein Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ—Ph, —S—(CH₂)ₘ—Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and
Y₂, Y₃, Y₄, and Y₅ independently represent hydrogen, alkyl(1-6C), alkoxy(1-6 C), haloalkyl(1-6 C), halo, NH2, NH-alkyl(1-6C), or NH(CH₂)ₙ—Ph wherein n is 0-3; or an adjacent pair of Y₂, Y₃, Y₄, and Y₅ form a fused 6-membered aromatic ring optionally containing up to 2 nitrogen atoms, said ring being optionally substituted by one o more substituents independently selected from alkyl(1-6 C), alkoxy(a-6 C), haloalkyl(1-6 C), halo, NH₂, NH-alkyl(1-6 C), or NH(CH₂)ₙ—Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ—Ph wherein n is 0-3; and
one of X₁ and X₂ is N and the other is NR₆, wherein R₆ is hydrogen or alkyl(1-6 C).

21. The method of claim 10 wherein said small organic molecule is a compound of formula (4) 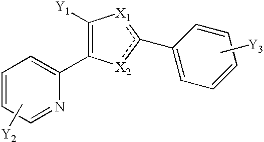
wherein Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)ₙ—Ph, —S—(CH₂)ₙ—Ph, cyano, phenyl, and CO₂R, wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ—Ph or NH-alkyl(1-6 C), wherein n is 0, 1, 2, or 3;
Y₃ is CO₂H, CONH₂, CN, NO₂, alkylthio(1-6 C), —SO₂-alkyl(C₁₋₆), alkoxy(C1-6), SONH₂, CONHOH, NH₂, CHO, CH₂NH₂, or CO₂R, wherein R is hydrogen or alkyl(1-6 C);
one of X₁ and X₂ is N or CR′, and other is NR′ or CHR′ wherein R′ is hydrogen, OH, alkyl(C-16), or cycloalkyl(C₃₋₇); or when one of X₁ and X₂ is N or CR′ then the other maybe S or O.

22. A method for the treatment of a subject having impaired lung function comprising administering to said subject an effective amount of a molecule capable of inhibiting a biological activity mediated by a TGFβ-R1 kinase receptor.

23. The method of claim 22 wherein said subject is human.

24. The method of claim 23 wherein said molecule specifically binds to said TGFβ-R1 kinase receptor.

25. The method of claim 24 wherein said impaired lung function is associated with a disease or condition selected from the group consisting of emphysema, chronic bronchitis, chronic obstructive pulmonary disease (COPD), pulmonary edema, cystic fibrosis, occlusive lung disease, acute respiratory deficiency syndrome (ARDS), asthma, radiation-induced injury of the lung, lung injuries resulting from infectious causes, inhaled toxins, or circulating exogenous toxins, aging and genetic predisposition to impaired lung function.

26. The method of claim 25 wherein administration is in the form of a pharmaceutical composition.

27. The method of claim 26 wherein said pharmaceutical composition is suitable for oral administration.

28. The method of claim 26 wherein said pharmaceutical composition is suitable for intravenous administration.

29. The method of claim 26 wherein said pharmaceutical composition is suitable for aerosol administration.

30. The method of claim 26 wherein said pharmaceutical composition is suitable for intrapulmonary administration.
